(19) United States
(12) Patent Application Publication
(10) Pub. No.: US 2025/0312310 A1
(43) Pub. Date: Oct. 9, 2025

(54) Beta2-adrenergic receptor antagonists for use in treating diabetes or obesity

(71) Applicant: NovelYeast bv, Oud-Heverlee (Blanden) (BE)

(72) Inventors: Johan Maria J Thevelein, Oud-Heverlee (Blanden) (BE); Chetan Pradeep Kulkarni, Leuven (BE); Walter Herman Maria Louis Luyten, Bertem (BE)

(73) Assignee: NovelYeast bv, Oud-Heverlee (Blanden) (BE)

(21) Appl. No.: 18/707,187

(22) PCT Filed: Nov. 8, 2022
(86) PCT No.: PCT/EP2022/081120

§ 371 Date: May 3, 2024

(30) Foreign Application Priority Data
Nov. 8, 2021 (EP) 21206861.3

(51) Int. Cl. A61K 31/405; A61P 3/04; A61P 3/10; G01N 33/50

(52) U.S. Cl. CPC A61K 31/405; A61P 3/04; A61P 3/10; G01N 33/5008

(57) ABSTRACT

Intestinal epithelial cells (enterocytes) sense the presence of sugars in the gut to stimulate sugar absorption, but the receptor has remained unknown. We show strong expression of the β2-adrenergic receptor (β₂-AR) at the enterocyte apical membrane and reveal its function in stimulating glucose uptake from the gut by the sodium/glucose-linked transporter, SGLT1. Upon heterologous expression, the β₂-AR responds to multiple sugars in the mM range, consistent with estimated gut sugar levels after a meal. Some adrenergic receptor antagonists differentially inhibit epinephrine and sugar responses. Glucose uptake into everted sacs from rat intestine was stimulated by epinephrine and sugars in a β₂-AR-dependent manner. STD-NMR confirmed binding of glucose to β₂-AR. Administration of glucose with a non-bioavailable β₂-AR antagonist lowered the subsequent increase in blood glucose levels, confirming a role for apical β₂-ARs in stimulating gut glucose uptake and suggesting enterocyte β₂-AR as novel drug target in diabetic and obese patients. Future work will have to reveal how glucose sensing by enterocytes and neuroendocrine cells is connected and whether β₂-ARs mediate glucose sensing also in other tissues.

### INTRODUCTION

Diabetes and obesity are two widespread diseases that could strongly profit from a precisely controlled reduction of sugar uptake from the gut. Extensive evidence has been reported that the main cells in the intestinal epithelium, the enterocytes, have mechanisms to sense the presence of sugar in the gut and respond by increasing sugar uptake from the gut lumen (1). Most of this uptake is mediated by the Na⁺-glucose linked transporter SGLT1, located in the apical membrane of enterocytes facing the intestinal lumen (2). Sugar-induced upregulation of SGLT1 activity and corresponding stimulation of glucose uptake from the gut is well-documented (3-6). A wide range of sugars, including non-permeable sugar analogs, is able to elicit the response, which requires no metabolism of transported sugars (7). Evidence was obtained that the sugar sensing involved a G protein-coupled receptor (GPCR) acting through the CAMP-Protein Kinase A (PKA) pathway (4, 5, 8). Sweet taste receptors were shown to be involved in sugar-induced stimulation of glucose uptake by enterocytes (9, 10) and carnivores lacking a functional sweet taste receptor, like cats, also lack glucose-induced stimulation of glucose transport in response to dietary sugar (11, 12). Later work revealed that expression of the sweet taste receptors is confined to certain intestinal neuroendocrine cells (representing less than 1% of intestinal epithelial cells) to regulate the release of incretin peptide hormones that somehow stimulate glucose uptake in the absorptive enterocytes (13, 14). How these peptide hormones control SGLT1 activity in the neighbouring enterocytes has remained unclear (15). Over 99% of intestinal epithelial cells are absorptive enterocytes that express SGLT1, whose activity is regulated by the glucose level in the gut (4). The increase in enterocyte glucose transport starts within seconds of exposure to sugars, which appears too fast for the proposed neuroendocrine loop (9), but rather suggests an additional cell-autonomous process in the enterocyte itself. Moreover, incretin secretion is also stimulated by lipids and proteins (16), which do not trigger increased glucose transport, suggesting an additional requirement for sugar regulation of SGLT1. Thus, there seems to exist an elusive sugar-specific sensor in the enterocytes regulating sugar uptake via control of SGLT1 activity.

We have previously discovered a glucose/sucrose-sensing GPCR, Gpr1, in the yeast Saccharomyces cerevisiae. It regulates energy and reserve carbohydrate metabolism through the CAMP-PKA signaling pathway (17, 18) in response to glucose and other rapidly-fermented sugars. Similar sugar-sensing receptors belonging to the same GPCR subfamily and also acting through the CAMP-PKA pathway have been found in Schizosaccharomyces pombe (19), Candida albicans (20, 21), Neurospora crassa (22), and homologs are present in many other fungi (23). The physiological function of the GPCR Gpr1 in S. cerevisiae resembles the regulation of energy and reserve metabolism in certain mammalian cell types, e.g. liver and fat cells, by the β2-adrenergic receptor (β₂-AR) in response to epinephrine (24, 25). The β₂-AR is a major drug target and likely the best-characterized mammalian GPCR (26, 27). Its three-dimensional structure has been determined for different agonist- and Gₛ-protein-associated states (28-32). The β₂-AR belongs to a family of closely related adrenergic receptors responsive to catecholamines, like epinephrine and norepinephrine (33). Antagonists and agonists with varying specificity for the different α- and β-adrenergic receptor subtypes have been identified, and many of those are in clinical use as drugs, e.g. in cardiovascular and respiratory medicine (27, 34-36).

Following our discovery of the yeast sugar-sensing GPCR Gpr1, we have explored the possible presence of a sugar-sensing GPCR in the apical membrane of mammalian enterocytes. Both cell types are exposed to highly variable nutrient conditions in their environment, and both also respond to glucose with specific GPCR- and cAMP/PKA-dependent responses controlling carbohydrate metabolism. As shown in the present paper, we surprisingly found that the β₂-AR is abundantly present in the apical membrane of enterocytes, a location more suggestive of a function as sensor for components like nutrients in the gut lumen, rather than for catecholamine sensing. The use of two different heterologous expression systems subsequently revealed that the β₂-AR responds to multiple sugars at concentrations (5-100 mM) found in the gut after a meal. Experiments with everted sacs prepared from rat intestine, and determination of blood glucose levels after in vivo administration of glucose together with a non-bioavailable β-AR antagonist confirmed that the β₂-AR mediates sugar-induced stimulation of glucose uptake from the gut lumen. The confirmation by STD-NMR of direct binding of glucose at mM concentrations to the β₂-AR, hints at a possible more universal role of the β₂-AR as glucose-sensing receptor in other mammalian cell types. Our results suggest that the β₂-AR evolved from an ancient sugar receptor in an ancestral primitive unicellular eukaryote, and has retained its sugar-binding capacity to function as a sugar-sensor in the gut and possibly in other mammalian tissues.

### Results

### The β₂-AR is Expressed at the Apical Membrane of Enterocytes.

We have first tested which GPCRs are expressed in enterocytes (Supplementary Table 1), in order to investigate subsequently those located in the apical membrane for a possible sugar-sensing function. We first used PCR amplification of selected GPCRs (37) starting from cDNA derived from mouse proximal duodenal scrapings, and from the intestinal STC-1 cell line. Twenty-five GPCRs could be amplified in this way from both sources (Supplementary Table 1). We then performed microarray gene expression analysis of STC-1 cells and SYBR Green qPCR for confirmation. We identified 22 GPCRs and one additional selected GPCR, not present on the microarray (Supplementary Table 1). Finally, we confirmed by qPCR the expression of 18 GPCRs using RNA from STC-1 cells (Supplementary Table 1).

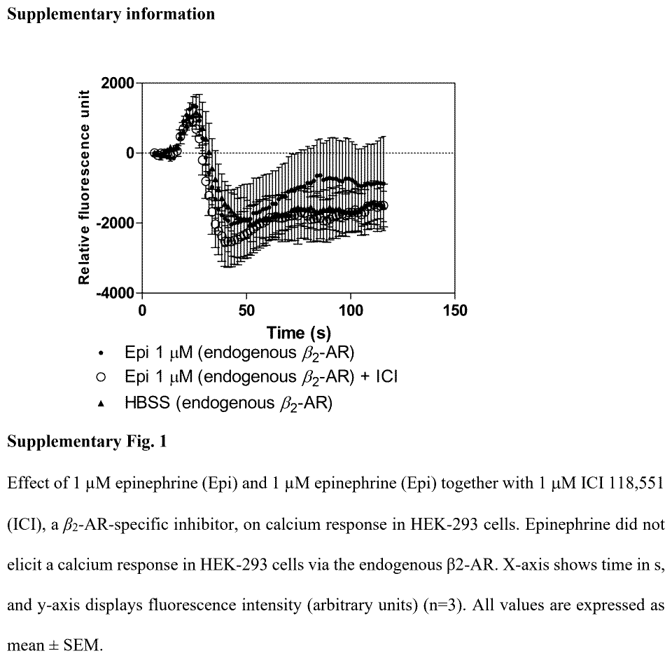

Next, we used immunohistochemistry on human intestinal biopsy samples to determine if any of the GPCRs were located in the apical membrane. We first tested GPCRs for which antibodies were available. Among the first GPCRs tested: GPR105, GPR1, GPR120, GPRC5C and the β₂-AR, only the last one surprisingly showed staining at the apical cell membrane, which was very strong and highly reproducible, compared to the much weaker staining with some of the other GPCRs (FIG. 1a). These either showed staining at the basolateral membrane or showed staining too weak for reliable detection. Some much weaker cytoplasmic staining on the basal side of the enterocytes was also observed for the β₂-AR, possibly in organelles of the secretory pathway (FIG. 1b). Because this location (facing the luminal side of the gut) is highly unusual for a receptor sensing a hormone distributed through the bloodstream and since the β₂-AR is involved in glucose and energy homeostasis, and couples to the CAMP-PKA signaling pathway, we further concentrated on the possibility that the β₂-AR might have an additional glucose-sensing function. The apical localization of β₂-ARs in enterocytes was confirmed with paraffin-embedded tissue sections of proximal mouse intestine (FIG. 1c), and showed near complete overlap with immunoreactivity for SGLT1, which is specifically located in the apical membrane (FIG. 1d,e). This, however, does not necessarily indicate that the two proteins would physically interact. Human and mouse β₂-AR show 87% sequence identity (https://www.uniprot.org/align/A20200503DA437993067D6F64326E5E763500BDE D0207523). Our immunohistochemistry results demonstrating the apical localization of the β₂-AR in intestinal epithelial cells are consistent with previous results in the literature. Singh et al. (2009) reported the mRNA expression level for β₂-AR in murine duodenal epithelial cells, as well as the strong enrichment of β₂-ARs by Western blotting in the apical brush border membrane compared to the total cell lysate. Both β₂-AR bands (monomer and dimer) were completely blocked in the Western blot using the immunising peptide, showing the specificity of the antibody (38). Similar results were reported for colonic mucosa (39).

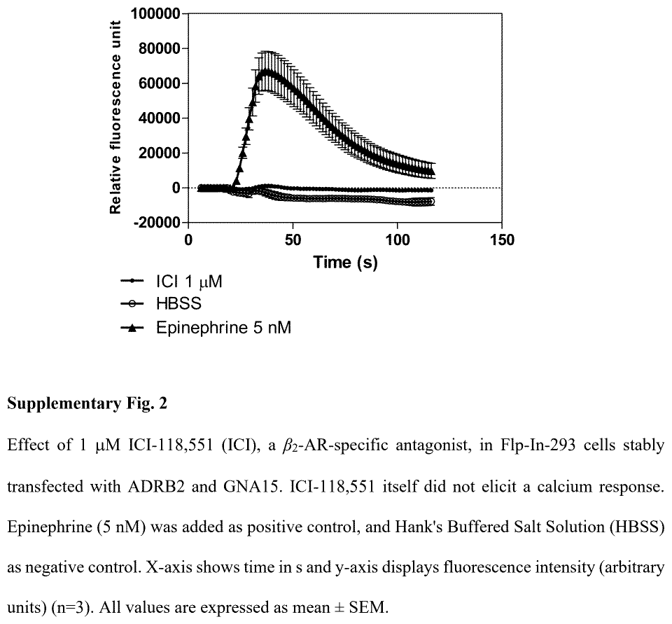

The β₂-AR responds to sugars and this response is blocked by β-AR antagonists. We subsequently constructed a stably transfected Flp-In-293 kidney cell line overexpressing a human β₂-AR, in which receptor activation is coupled via Gα16 to phospholipase-C-mediated Ca²⁺ release from intracellular stores, as detected by Fluo4 fluorescence. These cells responded as expected to catecholamine agonists, like epinephrine, and the response was blocked by classical β-AR antagonists (34, 35), like nadolol (β1,2-antagonist), labetalol (α,β-antagonist), propranolol (β1,2-antagonist) and ICI 118,551 (β₂-antagonist) (FIG. 2a). Control experiments with HEK293 cells without transfection of the β₂-AR failed to show a calcium response with epinephrine, indicating that any endogenous β₂-AR expression was too low to give a significant response with our reporter system (Supplementary FIG. 1). Addition of ICI 118,551 alone did not trigger a significant change in the fluorescence read-out, with epinephrine (5 nM) and Hank's Buffered Salt Solution (HBSS) used as positive and negative control, respectively (Supplementary FIG. 2). Using this reporter system, we next evaluated whether sugars were able to trigger activation of the β₂-AR. Glucose triggered a rapid, but less pronounced response compared to epinephrine, that was completely inhibited by the aforementioned β-AR antagonists (FIG. 2b). ICI 118,551 was equally effective in blocking the epinephrine and glucose responses at 30 μM. Other sugars also activated the β₂-AR in order of decreasing intensity: maltotriose, glucose, maltose, xylose, trehalose, fructose and 2 deoxy-D-glucose. all used at 50 mM (FIG. 2c), suggesting different degrees of agonist potency. Glucosamine (50 mM) provoked very little effect. ICI 118,551 inhibited all sugar responses at 1 μM. The sugar concentrations are in the same range as their estimated concentrations after a meal in the gut of mammals, including humans (40, 41). Rapid cell uptake of glucose at low concentrations hampered accurate determination of the apparent affinity of the β₂-AR for glucose. For maltotriose, a sugar not taken up by the cells, an EC₅₀ of ±10 mM was determined (FIG. 2d).

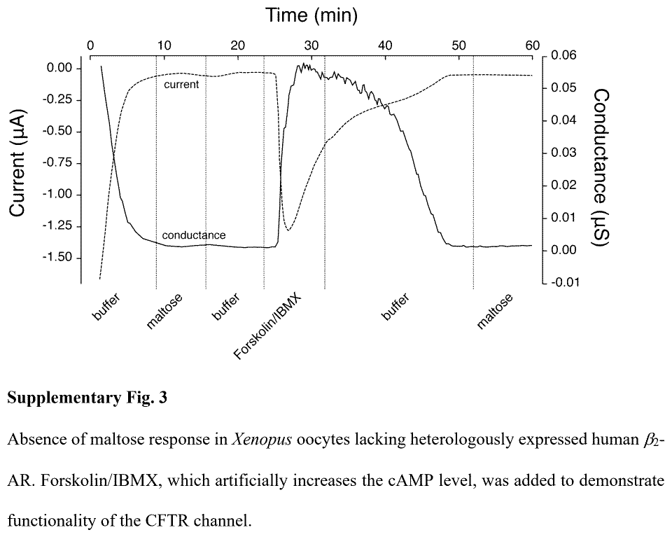

To exclude that the genetically engineered Gα16/phospholipase-C mediated Ca²⁺ signaling was in some way affecting the specificity of the β₂-AR, we also used a cAMP-dependent reporter system. For that purpose, we expressed β₂-ARs in Xenopus oocytes, together with a reporter system consisting of the PKA-dependent cystic fibrosis transmembrane conductance regulator (CFTR) Cl⁻ channel. Thus, parallel changes in inward transmembrane current and conductance of the membrane served as readout for receptor activity. Here as well, both epinephrine and different sugars activated the β₂-AR (FIG. 2e,f) while the β-AR antagonist propranolol inhibited the responses (FIG. 2e). There was neither an epinephrine nor a sugar response in control Xenopus oocytes injected with vector mRNA, while addition of forskolin (activator of adenylate cyclase) together with IBMX (inhibitor of cAMP phosphodiesterase) produced a strong response (Supplementary FIG. 3).

Sugars do not Compete with Antagonist Binding to β₂-AR.

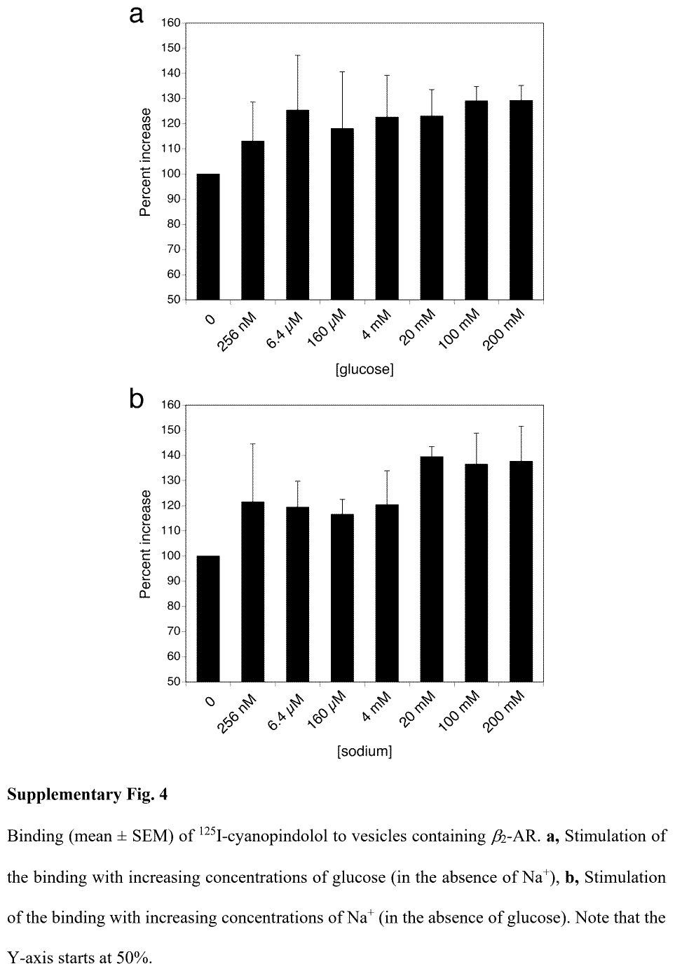

We next investigated whether the two types of agonists, epinephrine and sugars, bind to the same ligand-binding site. For that purpose, we determined binding of ¹²⁵I-cyanopindolol, a β-AR antagonist, to isolated cell-free membrane vesicles containing recombinant human β₂-AR (Perkin Elmer Cat. nr. RBHBE2M) in the absence or presence of glucose. We did not observe any inhibition of ¹²⁵I-cyanopindolol binding in the presence of glucose (Supplementary FIG. 4a), suggesting that the two compounds do not bind to the same site on the β₂-AR, in spite of our previous observation that the glucose response was blocked by a range of classical-AR antagonists (FIG. 2b). Surprisingly, we even observed an increase up to approximately 30% of ¹²⁵I-cyanopindolol binding with increasing glucose concentrations (Supplementary FIG. 4a). This suggests that glucose binding affects the structure of the epinephrine-binding site. The affinity of this glucose response, however, should not be confused with the affinity by which β₂-AR triggers activation of the Gₛ-protein upon binding of glucose alone. In that case indeed there is no other compound bound into the epinephrine binding site. The stimulation of cyanopindolol binding by glucose merely confirms that glucose physically interacts with β₂-AR although it also suggests that glucose could modulate epinephrine binding in vivo. We also tested the effect of glucose+NaCl because of a previous report that NaCl enhanced ¹²⁵I-cyanopindolol binding (42), which we also observed with increases up to roughly 35% (Supplementary FIG. 4b). Glucose+NaCl caused an increase up to approximately 40% (FIG. 3a). On the other hand, increasing concentrations of isoproterenol caused the expected gradual inhibition of ¹²⁵I-cyanopindolol binding (FIG. 3b). This appears to indicate that sugars and epinephrine may not bind precisely to the same site, although binding of the sugar does appear to affect the structure of the epinephrine binding site. This agrees with the demonstration of conformational coupling between the epinephrine binding site and an allosteric binding site for other ligands on the extracellular surface of the β₂-AR (43). Because both the epinephrine and sugar responses of the β₂-AR are inhibited by classical β-AR antagonists (FIG. 2a,b), the two binding sites may be located close to each other or may affect each other over a greater distance by the reported conformational coupling upon ligand binding. In recent years, an increasing number of allosteric ligands has been discovered for GPCRs. They bind to allosteric sites, as opposed to the orthosteric ligands that bind to the binding site for the native ligand (44). On the other hand, it is premature at present to suggest that glucose and adrenergic agonists would bind to two entirely different sites, or that glucose would bind to an allosteric site rather than the orthosteric site, especially because of the consistent inhibition of all sugar responses by β-AR antagonists.

### Some β-AR Antagonists Differentially Inhibit β₂-AR Responses to Epinephrine and Sugar.

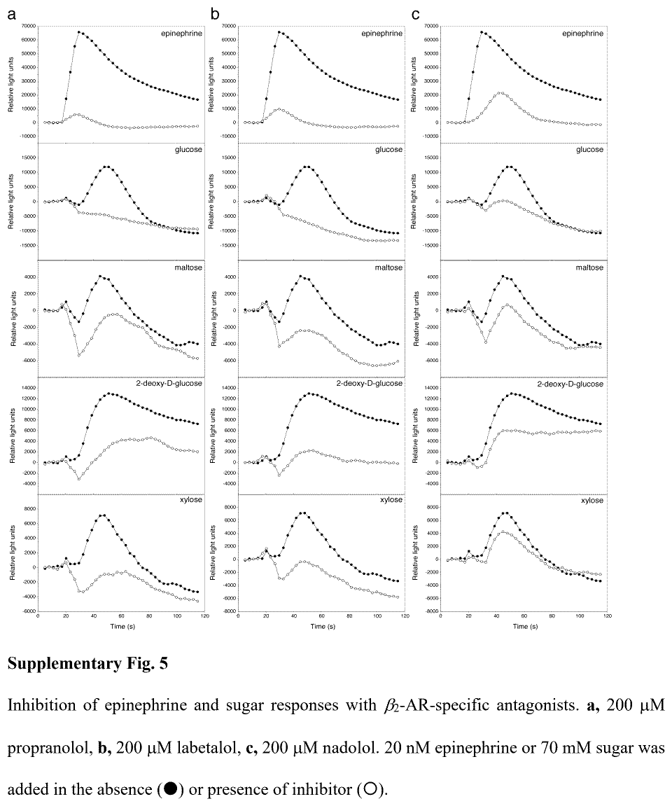

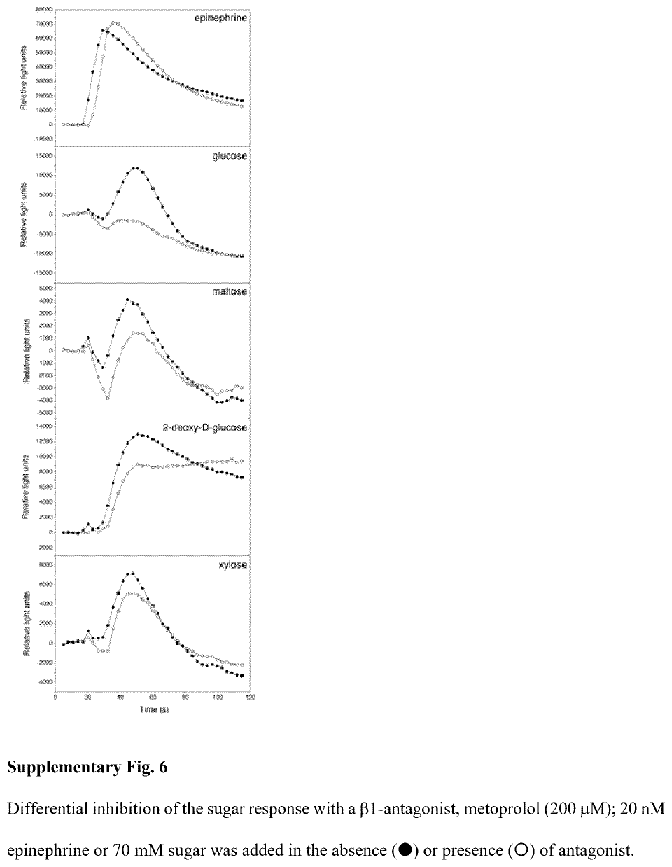

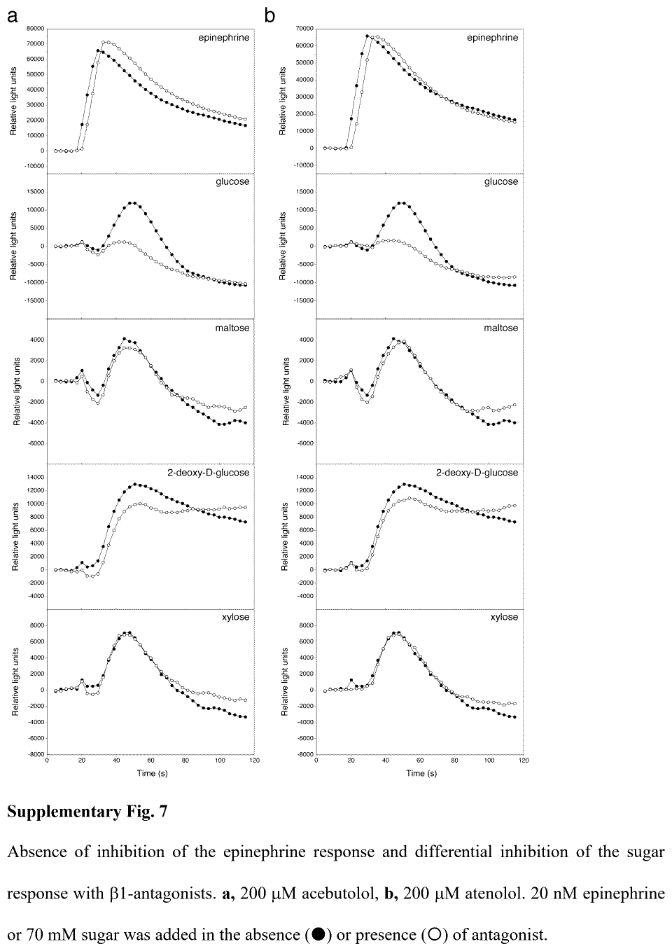

Because the two types of ligands, epinephrine and sugar, may bind to sites on the β₂-AR that differ at least to some extent, we tested several β-AR antagonists in search of compounds with a possible differential effect on the two ligand-binding sites. Responses to all sugars tested, i.e. maltotriose, glucose, maltose, xylose, trehalose, fructose and 2-deoxy-D-glucose were inhibited by β₂-antagonists: 1 μM ICI 118,551 (FIG. 4), propranolol, labetalol and nadolol (Supplementary FIG. 5). In contrast, β1-specific antagonists did not inhibit the epinephrine response (34, 35) (supplementary FIG. 6) but differentially affected the response to sugars. Metoprolol (supplementary FIG. 6), acebutolol or atenolol (Supplementary FIG. 7) at 200 μM completely inhibited the response to 70 mM glucose, but only slightly affected the response to 70 mM maltose, 2-deoxy-glucose or xylose (Supplementary FIG. 6; Supplementary FIG. 7). These results are consistent with binding of epinephrine and glucose to two different sites. The stronger antagonism of β1-antagonists for the glucose response, at least under the conditions of our experiments, compared to the other sugars suggests that glucose binds somewhat differently to the β₂-AR compared to the other sugars tested.

### Everted Sacs of Rat Intestine Show Sugar-Induced, β₂-AR-Dependent Stimulation of Glucose Uptake.

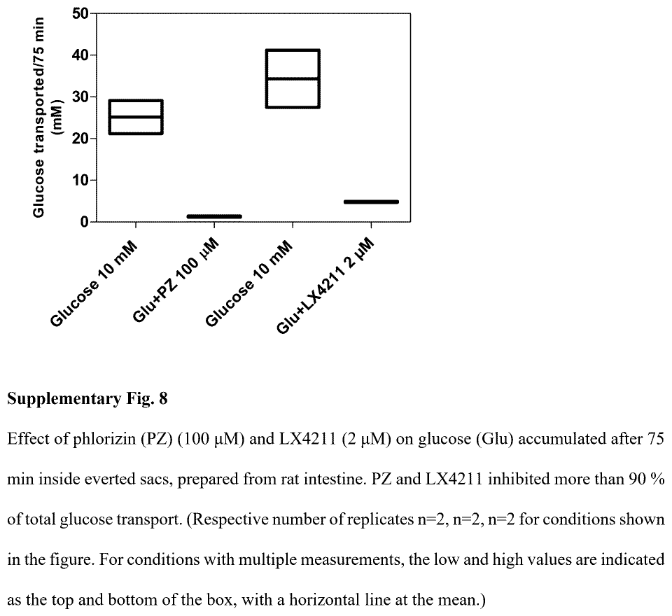

The importance of the apical β₂-AR for sugar-induced stimulation of glucose transport in the gut was evaluated with an everted sac model, in which facilitated glucose transport occurs from the external medium outside the sac, facing the intestinal mucosa, to the inside of the sac flanked by the serosa (45, 46). Glucose from the external medium (5 mM) indeed accumulated inside the everted sac at a rate that increased with longer incubation times (FIG. 5a). Glucose uptake into the everted sacs was inhibited for more than 90% by phlorizin (100 μM) and by LX4211 (2 μM), inhibitors of both SGLT1 and SGLT2 (Supplementary FIG. 8). When epinephrine (10 μM) was added to the external medium, the amount of glucose accumulated after 10 min in the sac increased from 440±132 to 833±255 μM. This increase was completely prevented by the β₂-AR antagonist ICI 118,551 (10 μM), while phlorizin (100 μM) inhibited both basal and enhanced glucose uptake (FIG. 5b). This supports a role of the apical β₂-AR in stimulating glucose uptake through SGLT from the gut. Colchicine (5 μM), a microtubule-disrupting agent that blocks translocation of SGLT1 from a cytoplasmic pool to the apical plasma membrane (47), completely blocked epinephrine stimulation of glucose transport, but not basal glucose uptake, as was observed with phlorizin (100 μM) (FIG. 5c). This suggests that epinephrine acts by increasing translocation of SGLT1 from a cytoplasmic pool to the enterocyte apical surface. Myristoylated PKI 14-22 amide (mPKI) (1 μM), a selective cell-permeable protein kinase A (PKA) inhibitor, partially inhibited epinephrine stimulation of glucose transport, as opposed to the complete inhibition by ICI 118,551 (FIG. 5d). The high mPKI concentration we used is known to cause complete inhibition of PKA in ex vivo preparations (48), and higher concentrations of mPKI also did not further reduce the response. If mPKI is able to cause complete inhibition of PKA in the epithelial cells of the everted sacs, these results would suggest that also PKA-independent signaling is involved in epinephrine stimulation of glucose transport through the β₂-AR.

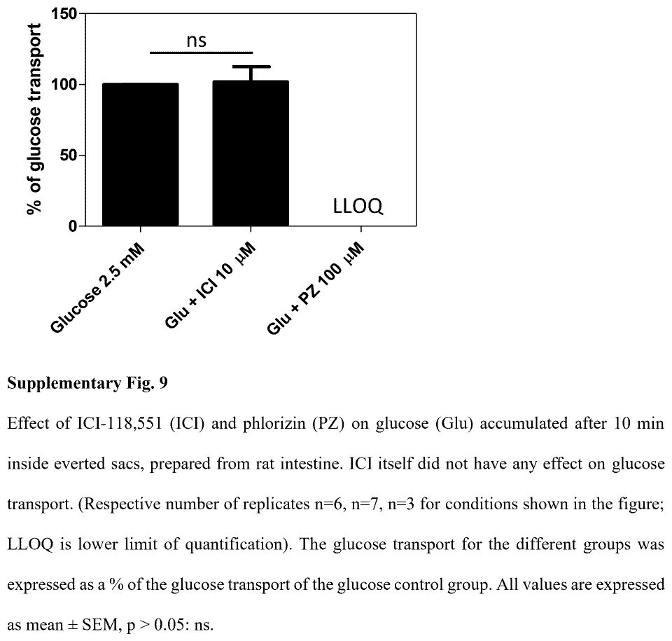

Next, we investigated whether sugar sensing by apical β₂-ARs could enhance glucose transport into the everted intestinal sacs. To avoid interference with glucose uptake by SGLT1 into the everted sacs, the β₂-AR was stimulated with mannose. Mannose is transported by SGLT4 but is not a substrate of SGLT1 (49, 50). Hence, any stimulation of glucose uptake through SGLT1 by mannose has to act through another target. We previously showed that mannose is also one of the sugars that potently stimulates the β₂-AR, as measured in stably transfected Flp-In-293 cells incubated throughout in 5 mM glucose, and ICI 118,551 completely blocked the mannose response (FIG. 5e). Five mM glucose or mannose equally potentiated the response of β₂-ARs to 5 nM epinephrine, compared to sugar-free medium (to which 4 mM glutamine was added as energy source). Whereas 5 nM epinephrine still stimulated the β₂-AR in sugar-free medium, although significantly less compared to medium containing 5 mM glucose or 5 mM mannose, mannose could only activate the β₂-AR in cells pre-incubated with glucose or mannose, and not in sugar-free medium (FIG. 5f). In everted sacs, both glucose transport and the stimulation of glucose transport by mannose were completely inhibited by LX4211, a dual inhibitor of SGLT1 and SGLT2 (FIG. 5g). The stimulation of glucose transport by mannose was abolished by the β₂-AR-antagonist ICI 118,551 (FIG. 5h), indicating that mannose stimulates glucose transport through β₂-ARs. Thus, sugar sensing by apical β₂-ARs in enterocytes stimulates glucose absorption through SGLT1. ICI 118,551 (10 μM) by itself did not have any effect on glucose transport in everted sacs (Supplementary FIG. 9). In this experiment, the everted sacs were incubated in a lower glucose concentration of 2.5 mM compared to the earlier experiments with 5 mM glucose, to reduce activation of β₂-ARs by glucose.

### Std-NMR Demonstrates Direct Glucose Binding to the β₂-AR.

To confirm direct binding of glucose to the β₂-AR, Saturation Transfer Difference (STD) NMR spectroscopy was performed with a membrane preparation containing the β₂-AR, derived from the transfected Flp-in-293 cell line, and a control membrane preparation, with minimal inherent expression of the β₂-AR, derived from the parent untransfected HEK293 cell line, similar to previous work showing direct binding of sugars to the human sweet taste receptor (51, 52). STD NMR has proven to be a powerful technique to study ligand binding to membrane proteins (53, 54). The STD spectrum of the β₂-AR-containing membranes in the presence of 10 mM glucose was very pronounced, while the spectrum of membranes lacking β₂-ARs but with the same glucose concentration had only a very small amplitude (FIG. 6). The difference STDD spectrum provides clear evidence for direct binding of glucose to the β₂-AR, since the saturation transfer can only take place upon physical binding of glucose to the target, the β₂-AR, which is the only different component between sample and control membranes. This suggests that glucose interaction is not restricted to apical β₂-ARs in intestinal epithelial cells, but that β₂-ARs in general can interact with glucose and thus may act as a glucose sensor (or at least respond to glucose) also elsewhere in the body.

Inhibition of Apical β₂-AR in Gut Epithelium with a Non-Bioavailable β-Antagonist Reduces Blood Glucose Levels after an Oral Glucose Bolus

Finally, we have performed in vivo experiments in which rats were orally administered a glucose bolus together with a non-bioavailable β-antagonist: CD3-403 (FIG. 7a). This compound was specially designed for our work based on the structure of ICI 118,551, which was modified by addition of a hydrophilic group and elimination of a hydrophobic group to ensure minimal permeability through biological membranes. This was confirmed by a Caco-2 permeability assay (Supplementary Table 2). CD3-403 inhibited with an IC₅₀ of 46 nM the calcium response elicited by 5 nM epinephrine in Flp-In-293 cells stably transfected with ADRB2 and GNA15 (FIG. 7b,c). The CD3-403 compound was synthesized at 100 mg scale for oral administration in rats (GVK Biosciences Private Limited). The glucose bolus (2 g/kg or 4 g/kg body weight) was administered by oral gavage to sets of three rats each, in the absence or presence of 5 μM (0.05 mg/kg) CD3-403. Glucose levels were measured in blood withdrawn from the tail vein just before, as well as 15, 30, 60 and 120 min after oral administration of the glucose bolus. The increase in blood glucose concentration after the glucose load is shown, normalized to the 0 min blood glucose concentration, which was set at 100%. In the case of glucose 2 g/kg, the presence of the CD3-403 compound caused a reduced increase in blood glucose level, which was significant at 60 min (FIG. 7d). For the experiment with glucose 4 g/kg, we also saw a trend of decreased glucose concentration after treatment with CD3-403 (FIG. 7e). For the experiment with glucose 2 g/kg, the area under the curve (AUC) was also calculated by setting 100% as a baseline. The CD3-403 compound significantly reduced the glucose increase (5907±2195; mean±SD) compared to the glucose 2 g/kg control group (8056±2990; mean±SD) (FIG. 7f).

### DISCUSSION

Our work provides strong evidence for an additional function of the β₂-AR as glucose receptor. Although the different results obtained might each be subject individually to more or less likely alternative explanations, the comprehensive body of evidence when taken together makes a compelling and consistent case that β₂-ARs in the apical enterocyte membrane sense the level of glucose in the gut to stimulate its uptake through SGLT1. This additional function is unexpected because the β₂-AR is one of the best-characterized GPCRs. It has served as a leading model for elucidation of the mechanisms involved in GPCR signaling, the three-dimensional structure of GPCRs and the conformational changes triggered by ligand binding (26, 28-32). As a major drug target, its pharmacology has been studied in great detail (27, 34, 35). It is the strong expression of β₂-ARs in the apical plasma membrane of the epithelial cells facing the lumen of the gut, a highly unusual location for a receptor sensing a hormone distributed through the bloodstream, that led us to the current discovery. Interestingly, strong expression of β₂-ARs in the apical membrane of the epithelium lining the proximal tubule of the nephron had already been reported (55), as well as stimulation of glucose reabsorption from the kidney tubules by epinephrine (56). This apical location was interpreted, however, to allow for a response to epinephrine leaking from the blood into the urine primary filtrate. Similarly, β₂-AR in the apical membrane of enterocytes might be activated fortuitously by epinephrine leaking through the tight junctions of the gut epithelium, although this is likely limited to pathological conditions given the lack of permeability in the tight junctions for small molecules like sugars and amino acids (57). Moreover, such explanations do not contradict our results demonstrating that enterocyte apical β₂-AR functions as a glucose receptor for stimulation of sugar uptake from the gut. Multiple studies on intestinal epithelial cells have reported results consistent with those in our work. Expression of β-AR subtypes in the gut has been demonstrated by [¹²⁵]cyanopindolol binding and its competition with adrenoreceptor antagonists (58). mRNA expression and apical membrane location of β₂-ARs was reported in a study on the regulation of apically located CFTR in murine duodenal epithelia (38). Moreover, β₂-AR agonists activate CFTR in intestinal organoids through cAMP signaling (59), and optimal activity of CFTR in Caco-2 human colon carcinoma cells is dependent on the presence of glucose (60). Coupling of the β2-AR to adenylate cyclase has been demonstrated in Caco-2 cell membranes based on the increase in cAMP level upon addition of different agonists (61).

The SGLT2, and to a lesser extent SGLT1, present in the apical membrane of the kidney tubule, are responsible for the reabsorption of glucose from the urine primary filtrate (62). Their expression is enhanced by luminal glucose (63) and their plasma membrane insertion and/or activity are enhanced by elevated cAMP-PKA signaling, as in intestinal (62, 64) and ovary (65) epithelial cells. Similar regulation of intracellular SGLT1 and SGLT2 trafficking to the plasma membrane both in small intestinal mucosa and kidney tubules by RSCIA1 (RS1) has also been reported (64, 66). Our results strongly suggest that β₂-ARs may sense glucose also in kidney tubule epithelium to stimulate glucose recovery from the urine primary filtrate. Such a role makes more physiological sense than regulation by leaked catecholamines in the primary filtrate.

Although the main acute effect of β₂-AR stimulation is an increase in blood glucose concentration due to glycogen mobilization in the liver and inhibition of glucose disposal by insulin-dependent tissues, there have been many reports on stimulation of glucose uptake in different tissues by activation of adrenergic receptors independently of insulin (67-77). In astrocytes, adrenergic stimulation of the β₂-AR acts through GLUT1 by coupling to Gₛ and activation of the adenylate cyclase pathway (78), while in rat skeletal muscle cells it causes increased translocation of GLUT4 to the plasma membrane (73, 79, 80). Our results show that stimulation of glucose uptake through activation of β₂-ARs does not only appear to target GLUT facilitated diffusion carriers in diverse somatic cell types, but also targets the active glucose transporter SGLT1 in intestinal epithelial cells. Involvement of the cAMP-PKA signaling pathway may be a common theme in adrenergic stimulation of glucose uptake.

It has been reported that knock-out mice in the sweet taste receptor TIR2+TIR3 or in gustducin lack the secretion of gut hormones triggered by dietary sugars and also lack sugar-induced upregulation of SGLT1 expression and glucose absorptive capacity (5, 10, 81), while intestinal sweet taste receptor stimulation upregulates SGLT1 (82). This sweet taste sensing system is expressed in enteroendocrine cells, and has to communicate with the enterocytes in which SGLT1 mediates bulk sugar uptake. Its inactivation apparently also disables the β₂-AR-based sugar-sensing system in the enterocytes identified in this paper. An explanation may be that the enteroendocrine cells regulate the sensitivity of the β₂-AR-based sugar-sensing system in the enterocytes, particularly over the longer term (hours to days). Inactivation of the enteroendocrine sweet taste sensing system may thus make the β₂-AR sugar-sensing system in some way insensitive. This could happen at different levels, such as sorting of SGLT1 to the apical membrane and/or post-translational modification of SGLT1 or any component in the signaling pathway from β₂-AR to the SGLT1 sorting mechanism. The increase of SGLT1-mediated glucose transport is based on different components with a divergent time-dependency, whose regulation may well be distinct (83). Within seconds after exposure to glucose, pre-existing SGLT1 are translocated from a cytoplasmic pool to the cell surface membrane of the enterocytes. Those surface transporters then accelerate their glucose transport activity, presumably due to phosphorylation as reported for SGLT1 in Chinese hamster ovary cells (65). Later, synthesis of new SGLT1 protein is induced. Finally, the basal SGLT1 expression level may change over days or weeks depending on the food composition, as has been observed when herbivores are weaned and switch from a milk to a grass diet (3, 4). Our study has focused on the short-term SGLT1 changes, whereas many studies of the enteroendocrine pathways have focused on longer-term effects.

GPCRs must be derived in evolution from proteins in unicellular organisms that lack the elaborate, extracellular endocrine signaling pathways present in higher eukaryotes, and nutrient-sensing GPCRs are prime candidates in this respect (84-87). Although members of the main GPCR families have been found in the most primitive metazoan organisms (85), the very poor sequence conservation between GPCRs makes it difficult to connect fungal and animal GPCRs in evolution (87). Our results suggest that β₂-ARs evolved from an ancient glucose receptor, an ancestral GPCR present in a primitive unicellular eukaryote, similar to the Gpr1 glucose-sensing GPCR present in yeast and other fungi (17, 18, 23). Since sequence conservation between GPCRs is very limited in general, and especially between distant relatives like the yeast and mammalian GPCRs (88), it is not possible at this point to make a meaningful prediction of a putative common glucose binding site. β₂-AR immunoreactivity is abundant in the pharynx rim of the unicellular protozoon Paramecium, and was proposed to function as a nutrient sensor (89). It undergoes isoproterenol-induced desensitization by endocytosis, possibly initiated through phosphorylation by a homolog of the human β-adrenergic receptor kinase (βARK2, GRK3) (90). Expression of a β₂-AR homolog in Paramecium was confirmed by RT-PCR and Northern blot analysis (91), and several adrenergic receptor orthologous genes have been annotated as such in the Paramecium genome (https://paramecium.i2bc.paris-saclay.fr/cgi/tool/search). Nascent phagosomes are formed at the pharynx rim, and β-adrenergic agonists stimulate phagocytosis in Paramecium, a response blocked by β-AR antagonists, and potentiated by forskolin, an activator of adenylate cyclase (92, 93). Our results suggest that the Paramecium β₂-AR homolog may indeed function as a sugar sensor, consistent with its localization at the pharynx rim and its involvement in triggering phagocytosis. Radioligand binding studies also provided evidence for the presence of β-AR s in the unicellular protozoon Trypanosoma cruzi (94).

The STD-NMR results provide clear molecular evidence that glucose directly binds to the β₂-AR, since the saturation transfer can only take place upon physical binding. The lack of competition between glucose and cyanopindolol for binding to the β₂-AR (FIG. 3a, Supplementary FIG. 4a) suggests that the binding sites are different. On the other hand, the inhibition of the glucose-induced response by classical β₂-AR antagonists (FIG. 2b) and the stimulation of cyanopindolol binding to the β₂-AR by glucose (FIG. 3a, Supplementary FIG. 4a), indicates that binding of a molecule into one of the two binding sites affects the other binding site and that therefore the two binding sites might be in close interaction and possibly close proximity. Further analysis of sugar binding affinity and specificity with purified β₂-ARs and determination of the precise binding site in relation to that of β₂-AR agonists and antagonists, goes beyond the scope of the present work, but should be a major focus in future research. This should also include the precise interaction of β₂-AR with Gₛ proteins in response to glucose and epinephrine, as well as the possible sugar-sensing function of the other members of the β-AR family. In the glucose-sensing yeast GPCR, Gpr1, evidence was reported for direct interaction of glucose with TMD VI (18), a transmembrane domain involved in binding of small ligand molecules in many GPCRs, including the β₂-AR (95). During its evolutionary development into a hormone receptor, the β₂-AR has apparently retained its ancient glucose-sensing function. The unexpected localization of β₂-ARs in the apical membrane of intestinal epithelial cells, making little physiological sense for a hormone receptor, has enabled us to identify this function. The β₂-AR is used in enterocytes to sense luminal sugar and regulate its uptake from the gut. Interestingly, both the β₂-AR and yeast Gpr1 use the CAMP-PKA signaling pathway to control storage carbohydrate levels and sugar catabolism (96). Our results also explain previous observations that perfusion of rat small intestine with epinephrine significantly increases transport of glucose from the gut lumen to the blood (97, 98). This is associated with an increase in SGLT1 protein and phlorizin binding, and was also elicited by perfusion with dibutyryl-cAMP (98). Epinephrine is a water-soluble compound with a very low membrane permeability coefficient of 2.7=1.5×10⁻⁶ cm/sec (99), which precludes any significant passive diffusion through membranes. Hence, epinephrine should in principle not be able to reach the baso-lateral membrane of the epithelial cells when administered in the gut lumen. Aschenbach et al. (100) reported stimulation of SGLT1-mediated glucose uptake in isolated ovine ruminal epithelia by several β₂-AR agonists. Stimulation by forskolin, an activator of adenylate cyclase, and inhibition by the PKA inhibitor H89 supported involvement of cAMP-PKA signaling. These observations are consistent with the presence of β₂-ARs in the apical membrane, and cAMP signaling as mediator of enhanced SGLT1 expression and glucose uptake as a result of β₂-AR stimulation. Our discovery that the β₂-AR can function as a sugar receptor on the apical side of the intestinal epithelial cells provides a logical explanation for upregulation of SGLT1 and glucose uptake elicited by perfused epinephrine. Future research should study in more detail the molecular mechanisms involved in β₂-AR-mediated upregulation of SGLT1 at the transcriptional and post-translational level, including SGLT1 intracellular trafficking and ligand-induced endocytosis, as well as the composition of the signaling pathway(s) involved and their possible interaction with incretin hormones, and other mechanisms of neuroendocrine signaling.

It would be useful to complement the present work with genetic experiments in mice lacking the β₂-AR and testing for the rate of glucose transport from the gut to the blood. However, given the complex role of the β₂-AR in glucose homeostasis in the mammalian body, acting in different ways on glucose mobilization from reserve tissues and glucose sequestration in other tissues, it would be imperative to perform such genetic experiments with tissue-specific knock-out mice lacking the β₂-AR specifically in the intestinal epithelium. Otherwise, interpretation of the results would likely be very cumbersome and inconclusive. On the other hand, we would like to emphasize that the use of a non-bioavailable beta-blocker to specifically inhibit the sensing of glucose by the β₂-AR in the gut epithelium is a much stronger and more reliable scientific argument than the use of a tissue-specific knock-out because of the many possible complications that the latter may cause, including upregulation of related adrenergic receptors, abolishment of regular β₂-AR interactions and possible adverse effects on intestinal epithelial cell development, as well as the general shortcoming that elimination of a physiological response by deletion of a receptor protein does not necessarily imply that the sensing function of the receptor is directly responsible for the absence of the response.

Another aspect that is likely different between β₂-ARs in the apical membrane of enterocytes compared to the plasma membrane of other cell types is the degree and nature of post-translational modification, in particular glycosylation. The extracellular domains of apical membrane proteins in enterocytes are uniquely and heavily glycosylated (101). This glycosylation changes during their differentiation as they migrate upwards from the crypt base to the villus tip, and it is also influenced by the composition of the gut content (102). Glycosylation often affects protein functionality (103) and future research thus will have to determine whether changes in β₂-AR glycosylation affect the affinity and/or specificity of the sugar-sensing function of apical β₂-AR in the enterocytes.

Reduction of glucose uptake from the gut by SGLT1 inhibitors is being explored for treatment of diabetes and obesity (104, 105). Enhanced uptake of glucose from the gut by upregulation of SGLT1-activity was shown to cause obesity in mice (106). The expression of SGLT1 and other sugar transporters was found to be upregulated in gut epithelium from diabetic patients, suggesting that an increased capacity to absorb sugars from the gut may reinforce diabetic syndromes (64, 107, 108). Similar findings were made for obese patients (109). Apical β₂-ARs in enterocytes may constitute a more attractive target than SGLT1 for partially blocking postprandial glucose uptake from the gut in diabetic and obese patients, since antagonism of these β₂-ARs only blocks the sugar-induced stimulation of glucose uptake and not total glucose uptake, as is the case with SGLT1 antagonists. The latter easily results in osmotic diarrhea, enhanced microbial activity and flatulence, as a consequence of excessive gut sugar levels (110, 111). Obviously, β₂-AR antagonists with limited oral bio-availability would be the drugs of choice for this purpose, so as to avoid interference with the β₂-AR in other tissues of the body. Our work has now shown that a non-bioavailable β₂-AR antagonist is able to reduce the increase in blood glucose level when administered together with an oral glucose bolus. This suggests that non-bioavailable β₂-AR antagonists could be useful in humans to lower the postprandial increase in blood glucose level, and therefore might be used to reduce glucose uptake in diabetic and obese patients. It has been reported previously that in an intraperitoneal glucose tolerance test in β₂-AR knock-out mice (112) or after introducing an intravenous blood glucose load together with a β₂-AR antagonist in rats (113) or diabetic patients (114), a much higher increase in blood glucose level was observed compared to controls. This supports our conclusion that the reduction in blood glucose level observed in our in vivo experiments cannot be due to systemic inhibition of β₂-ARs, but must rather result from inhibition of the apical β₂-ARs in the gut epithelium. Future research should study the effect of administration of non-bioavailable β₂-AR antagonists as well as agonists in different nutrient regimes on glucose homeostasis, body weight gain and levels of insulin, glucagon, ghrelin, GLP-1 and GIP incretins, and other hormones known to be linked to glucose homeostasis in the body, in healthy individuals as well as in diabetic and obese patients.

We have used experimental conditions that in our view would maximize the chance of detecting a significant effect on the blood glucose level by administration of a non-bioavailable β₂-AR antagonist. However, it is unclear what the main driving force was in evolution to establish and maintain a glucose receptor in the gut to stimulate glucose uptake. Was it to maximize high glucose uptake during sparse meals? Or was its main function to stimulate uptake of low glucose levels under malnutrition conditions? Future research will have to investigate the effect of non-bioavailable β₂-AR antagonists under a variety of feeding conditions, not only on blood glucose levels, but also on general glucose homeostasis and other relevant parameters, such as body weight gain.

Glucose-induced regulatory effects are quite common in mammalian tissues, which is not surprising in view of the crucial role of glucose as a nutrient throughout the body. It is presently unclear whether the β₂-AR may also serve as glucose receptor for regulation of glucose-controlled processes in cell types or tissues other than those investigated in this study. The glucose-sensing function of the β₂-AR might play a role in multiple ways in the complex regulatory network controlling glucose homeostasis in the body. For instance, glucose sensing by β₂-ARs may modulate epinephrine sensing in blood and other body fluids, as suggested by the glucose stimulation of [¹²⁵I]cyanopindolol binding to the β₂-AR (FIG. 3a, Supplementary FIG. 4a), or may support feedback inhibition of β₂-ARs by high glucose levels through desensitization, stimulation of its endocytosis and/or prevention of its recycling (115, 116).

The glucose-sensing function of the β₂-AR may provide an explanation for some of the hitherto unexplained observations in human (patho)physiology related to β₂-AR function, such as unexpected negative side effects of β-AR antagonist therapy (117, 118) or unexplained differences in therapeutic outcomes between different β-AR antagonists (119, 120). The abnormally high glucose levels in diabetic patients may cause spurious activation (or desensitization) of β₂-ARs throughout the body, and be responsible for hitherto unexplained symptoms and complications of diabetes, like the well-established correlation between diabetes, hypertension and heart failure (121). Although epinephrine is well known to increase blood glucose levels through stimulation of glycogen breakdown, this appears to be a short-lived effect. In the long term, epinephrine increases muscle glucose uptake (122, 123). Stimulation of glucose uptake by epinephrine has been documented in muscle upon chronic administration (124) and also in brown adipose tissue (125). Stimulation of glucose uptake by β₂-ARs may be relevant in other cell types as well. Particularly cells in which very active glucose uptake is critical, e.g. in cancer cells where the reasons for the strong correlation between β₂-AR expression and cancer aggressiveness (126), the frequent involvement of β₂-ARs in multiple carcinogenic processes (127), as well as the beneficial effect of β-AR antagonists on the recovery of cancer patients during chemotherapy (128), have remained enigmatic up to now. The β₂-AR has also been reported to show basal ligand-independent activity, whereas this property is considerably weaker in the closely related β₁-AR subtype (129). Since glucose is present in most experimental media and body fluids, this sugar may have contributed to the basal “ligand-independent” activity, particularly since ICI 118,551 (which blocks the β₂-AR response to glucose) can block this spontaneous activity (129).

In conclusion, we have discovered that the β₂-AR, a well-established catecholamine receptor, is located at the apical side of intestinal epithelial cells to serve as a sugar sensor for stimulation of glucose uptake by SGLT1 from the lumen of the mammalian gut. We demonstrate direct binding of glucose at physiological concentrations to recombinant β₂-ARs contained in membrane vesicles, suggesting that the β₂-AR may be able to exert a more general glucose-sensing function also in other cells and tissues, and in other organisms.

### Materials and Methods

PCR techniques. The templates used were RNA extracts from sheep mucosal scrapings and from STC-1 cells, isolated at the laboratory of Prof. Shirazi-Beechey (University of Liverpool), sent on dry ice and stored at −80° C. First-strand cDNA synthesis was achieved using the RevertAid™ H Minus First Strand cDNA synthesis kit (Fermentas) according to the manufacturer's protocol, using 5 μg of purified RNA. Both oligo (dT) and random hexamer primers were used in the reaction. The resulting cDNA was stored at −20° C. Alternatively, the Thermoscript® RT kit (Invitrogen) was used for cDNA synthesis according to the supplied protocol. PCR primers are listed in Supplemental Information (Supplementary Table 3).

Quantitative PCR analysis was performed with SYBR® Green. Reactions were initiated with 100 ng of cDNA, 300 ng of both primers and the SYBR green MasterMix (Eurogentec) in a total reaction volume of 25 μL. Between 40-50 amplification cycles were performed in the ABI Prism® 7000 apparatus (Applied Biosystems).

Micro-array genome-wide gene expression analysis. RNA from STC-1 cells, cultured either in the presence of 5 or 25 mM glucose for the last 24 h, was used as template. First-strand cDNA synthesis was achieved using the RevertAid™ H Minus First Strand cDNA synthesis kit (Fermentas) according to the supplied protocol, using 5 μg of purified RNA. Both oligo (dT) and random hexamer primers were used in the reaction. The resulting cDNA was cleaned using the QIAquick PCR Purification Kit (Qiagen), and was used by the VIB Micro-Array Facility (VIB MAF vzw, Leuven, Belgium) for whole-genome expression analysis with Affymetrix GeneChip® Mouse Genome 430 2.0 Arrays.

Immunohistochemistry. Mouse and human tissues were embedded in paraffin after formaldehyde fixation. Both tissue samples were taken from the duodenal part of the small intestine. Anti-hußβ₂-AR (1/100) (Abcam) was used as primary antibody for peroxidase staining on human tissue. We also detected β₂-AR in control tissues known to express this receptor using the same antibodies. Secondary antibodies were peroxidase-labeled goat anti-rabbit antibodies (Abcam). The stained tissues were developed with 3,3′-diaminobenzidine, and they were counterstained with hematoxylin (Gill III). Antibodies used for fluorescent staining and co-localization in mouse tissue were rabbit anti-β₂-AR (Assay Design) (1/50) and goat anti-SGLT1 (M19, Santa Cruz) (1/50). Secondary antibodies used were anti-rabbit immunoglobulin labeled with Cy3 (Jackson) and anti-goat immunoglobulin labeled with FITC (Jackson). Cells were counterstained with DAPI. For imaging, Zeiss 63x oil immersion was used, and photographs were made of the human samples with a Zeiss Axioplan 2 microscope using Axiovision software. For the mouse samples, a Hamamatsu Orca AG microscope was used with Smart Capture Software.

Two-Electrode Voltage Clamp (Xenopus oocytes). The cDNA sequence of a human β₂-AR (Missouri S&T cDNA Resource Center) was subcloned from the pcDNA3.1+ to the pGEMHE Xenopus expression vector (kind gift of Prof. Jan Tytgat, Leuven). The pGEMHE vector was cut with BamHI (New England Biolabs) and HindIII (Roche). The ADRB2 coding region was amplified with primers containing the aforementioned restriction sites, the amplicons were purified from a TAE-agarose gel, and cut with the same enzymes. The resulting product was ligated using the Ligafast™ Rapid DNA Ligation System (Promega) and the constructs were verified by sequencing (VIB Genetic Service Facility, University of Antwerp). They were cut with NheI (Roche) for linearization. The gene encoding the FLAG-tagged Cystic Fibrosis Transmembrane conductance Regulator (CFTR) ion channel was derived from the M2 901/pBQ4.7 vector (kind gift of Prof. Jan Eggermont, Leuven) and was linearized with XhoI (New England Biolabs) for in vitro transcription. RNA was then transcribed in vitro with the Ribomax kit (Promega) or the mMESSAGE mMACHINE T7 kit (Ambion) following the manufacturer's protocol. RNA was visualized and its quality assessed on RNase-free agarose gels, and stored at −80° C. Buffers used were Ringer's buffer (ORi): 90 mM NaCl, 3 mM KCl, 2 mM CaCl₂), 5 mM HEPES, adjusted to pH 7.6 with NaOH, and a high potassium buffer: 5 mM NaCl, 65 mM KCl, 2 mM CaCl₂), 5 mM HEPES and an equivalent amount of sugar or N-Methyl-D-glucamine (NMDG), ensuring equi-osmolarity between sugar-containing and sugar-free buffers. The maximum measured difference in osmolarity between the buffers was 3.6%.

For oocyte collection, female Xenopus frogs were anesthetized by submersion in ice water/crushed ice for 30 min. After abdominal incision, the ovarian lobes were pulled out, cut off, and the oocyte clump was placed in ORi. Oocytes were then liberated in ORi-collagenase (1 mg/mL) for 1-2 h on a shaking incubator, and subsequently transferred to a Ca²⁺-free solution using one washing step. They were shaken vigorously for maximum 15 min before collecting the individual oocytes in ORi buffer. Stage V oocytes were then selected under the microscope.

For injection of the oocytes, RNA encoding the CFTR and/or β₂-AR protein was drawn from a droplet afloat in mineral oil with a glass capillary. Injection volume was 46 nl. The amount of RNA varied between 10 and 25 ng. Injected oocytes were stored in ORi at 16° C. for maximum 3 days until ready for testing. During the measurements, oocytes were continuously clamped to −60 mV. All measurements were recorded using the home-made software, DSPOOC.

Assessment of β₂-AR responses in cultured mammalian cells. A cDNA for the human ADRB2 and GNA15 was purchased from the Missouri S&T cDNA Resource Center and used for the construction of a stable Flp-In-293 (a derivative of the parent HEK293 cell line, Invitrogen) β₂-AR/Gα16 cell line. The cell line expressing both β₂-AR and Gα16 was selected in two successive transfection steps. First, Flp-In-293 cells were co-transfected with pMET7-ADRB2-FRT and pOG44. After Flp recombinase-assisted stable integration, an isogenic cell pool expressing the β₂-AR was selected in hygromycin-containing medium (100 μg/ml). Second, the isogenic cell pool was cotransfected with p-GNA15 and pIRES-Puro2 (Clontech) in a 5:1 ratio, followed by selection of single clones in medium containing puromycin (0.8 μg/ml). Flp-In-293 cells were maintained in DMEM medium (Gibco), containing penicillin/streptomycin (Sigma) and fetal bovine serum (Sigma) (10%). Hygromycin (100 μg/mL) was added for cells containing the ADRB2 construct, and puromycin (1 μg/mL) was added for the cells containing the GNA15 construct. The Flp-In-293 cells were transferred for β₂-AR assays to clear- and flat-bottom 96-well plates in DMEM (Gibco) containing dialyzed fetal bovine serum (Sigma). The plates were coated with fibronectin (Sigma). For that purpose, the fibronectin solution was diluted 40 times in PBS, added in the plate wells (60 μL), and removed again after incubation for 1 h, after which the plates were allowed to dry for 1 h. The Fluo-4 NW kit (Invitrogen) was used for detection of calcium signals following the manufacturer's protocol. All compounds tested were dissolved in HBSS (Sigma) at 37° C. Adrenergic antagonists (Sigma) were always added together with the Fluo4 compound, 50 min before addition of agonist. Agonists were added just prior to the actual measurement of the calcium signals generated. The Flexstation II apparatus (Molecular Devices) was used to perform calcium measurements. After overnight growth of the cells in transfer medium in the aforesaid multiwell-96 plates, the medium was removed, the cells incubated with 100 μL of the loading dye solution (Fluo-4), the plates covered with aluminum foil, and incubated at 37° C. for 30-50 min. Then, 50 μL of a 3× concentrated agonist solution (sugar or epinephrine) was added just prior to the measurement in the Flexstation II, to give a total volume of 150 μL per well.

Response to epinephrine and mannose in Flp-In-293 cells stably transfected with ADRB2 and GNA15, incubated in different HBSS-based buffers. This experiment was performed for checking the response to mannose in different conditions. Cells were incubated with Fluo-4 in different HBSS-based buffers either containing glucose 5 mM, mannose 5 mM or without glucose but supplemented with 4 mM L-glutamine (HBSS composition (mM): 1.26 CaCl₂, 0.49 MgCl₂, 5.33 KCl, 0.4 MgSO₄, 0.44 KH₂PO₄, 137.9 NaCl, 0.34 Na₂HPO₄).

Competitive radioligand binding assay. β₂-AR-containing membranes (RBHBE2M) and ¹²⁵I-cyanopindolol (NEX189) (spec. act. 2200 Ci/mmol) were purchased from PerkinElmer. TRIS-HCl buffer containing MgCl₂ and EGTA was used as incubation and assay buffer, while TRIS-HCl buffer was used as wash buffer. All buffers were cooled and all further steps performed on ice. Membranes were diluted 150 times in assay buffer. The radioligand was diluted to a final concentration of 0.097 nM and the tested compounds were added at the indicated concentrations. The mixtures were incubated for 1 h at ambient temperature, and subsequently filtered using a vacuum pump over GF/C filters (Whatman), pre-soaked in 0.5% polyethyleneimine. Following 9 rinses with ice-cold wash buffer, the filters were transferred to vials, which were read in a gamma-counter (Gamma master LKB Wallac 1277). Non-specific binding of ¹²⁵I-cyanopindolol was determined with membranes devoid of β₂-AR.

Animals and diet. All the experiments were approved by the ethical committee on animal experimentation of the KU Leuven. Wistar rats (Harlan Netherlands B.V.) were maintained on a 12 h light-12 h dark cycle, and fed with standard rat food chow and water ad libitum.

Preparation of everted intestinal sacs. Rats weighing around 150 g were fasted overnight before the experiment, and euthanized by cervical dislocation on the day of the experiment. The abdomen was opened by a midline incision, and a segment of around 35 cm of proximal intestine was isolated. The intestinal segment was rinsed with ice-cold Ringer solution (composition (mM): 140 NaCl, 5 KCl, 1 MgCl₂, 2 CaCl₂, 10 HEPES, 10 TRIS, gassed with 95% O₂ and 5% CO₂, pH 7.4), and 4-7 everted sacs were prepared, each approximately 3 cm in length, by tying off the ends of the intestinal segments with threads (46). The sacs were filled with Ringer solution containing 5 mM mannitol (Sigma Aldrich) (to maintain osmotic balance) and L-glutamine (2 mM) (Sigma Aldrich) (as an energy source). Each everted sac was transferred to a separate glass beaker containing 50 mL continuously oxygenated Ringer solution with L-glutamine (2 mM), and maintained in a water bath at 37° C. Then, epinephrine 10 μM (Sigma Aldrich), mannose 5 mM (Sigma Aldrich), ICI 118,551 10 μM (Sigma Aldrich), phlorizin 100 μM (Sigma Aldrich), LX4211 2 μM (MedKoo Biosciences, Inc. USA.) or Ringer solution were added to the respective beaker for a 15-min pre-incubation (concentrations indicate final concentrations of the compounds). For the sacs treated with ICI 118,551, phlorizin or LX 4211, the inhibitors were added before mannose or epinephrine. After pre-incubation, 5 mM glucose, or 2.5 mM glucose for the ICI 118,551 control experiment, was added to the external buffer to initiate glucose transport. After 10 min incubation, a sample was collected from inside the sac using a 1 mL syringe (Terumo) with a 26 gauge needle (Terumo). These samples were analyzed using a glucose assay kit based on glucose oxidase (Sigma Aldrich, for details, see below). Sacs treated with colchicine 5 μM (SERVA Feinbiochemica) or myristoylated PKI 14-22 amide 1 μM (Tocris), were pre-incubated for an additional 10 min before adding epinephrine or ICI 118,551. Also for colchicine, a TRIS-free Ringer solution was used, as TRIS interferes with colchicine activity (130). For comparing glucose transport rates, each everted sac was first pre-incubated for 15 min in Ringer solution, then glucose was added to a final concentration of 10 mM for a further incubation during 5, 10 or 75 min. We noticed that it was important to take certain precautions during these experiments: the intestine was never allowed to overfill and be stretched while rinsing the intestinal lumen at the time of isolation and filling the everted sac with Ringer solution. Trapping air bubbles inside the everted sac was avoided. Also, the regions of the intestine where mucus was still present were not used for preparing everted sacs.

Glucose assay. The glucose concentration of the samples was assayed by a glucose assay kit (Sigma Aldrich, GOD/POD method). The assay procedure recommended by the kit was modified for small sample volumes as follows. Fifty μL of sample was transferred to a well of a 96-well plate, and at time zero, the reaction was started by adding 100 μL of assay reagent to the first well, and mixing. Each well was allowed to react for exactly 30 min at 37° C. The reaction was stopped by adding 100 μL of 12 N H₂SO₄ (Merck) into each well, and carefully mixing. The absorbance was measured at 540 nm with a plate reader (Tecan 200). Mannose is only detected by the glucose oxidase assay with about 100-fold lower sensitivity than for glucose (131).

### In Vivo Oral Glucose Tolerance Test

The oral glucose tolerance test was performed on normal rats weighing around 250 g, which were fasted for 16 h before the test. Blood was sampled by tail vein puncture, and the blood glucose level was measured by glucometer (Verio OneTouch glucometer). Rats were divided in four groups: one with glucose 2 g/kg or glucose 4 g/kg (Sigma Aldrich), both in the presence or absence of CD3-403. The CD3-403 compound displays very low cell permeability (assessed by a Caco-2 permeability assay), and hence we aimed for a local intestinal concentration of 5 μM, based on 10 times the IC₅₀ and using an additional safety factor of 10 for any non-specific binding to intestinal content such as mucus. Glucose or glucose along with CD3-403, dissolved in 0.9% saline, was administered to rats by oral gavage in the respective groups. Blood glucose was measured at 0, 15, 30, 60 and 120 minutes. All experiments were conducted around the same time in the morning.

### Statistical Analysis

For the experiments on epinephrine and mannose stimulation of glucose transport and their inhibition by ICI 118,551, and for the experiments on the effect of LX 4211 and phlorizin on glucose and mannose transport, a one-way ANOVA was performed, followed by Tukey's test. For the experiment on the effect of colchicine on epinephrine stimulation of glucose transport, the direction of the effect of colchicine (inhibition) and epinephrine (stimulation) was known, hence, a one tailed t-test was applied. For the effect of epinephrine and mannose in the calcium assay experiment, the epinephrine and mannose groups were analyzed individually by one-way ANOVA, followed by Tukey's test. For the effect of ICI 118,551 on glucose transport, a two-tailed t-test was applied. In case of the in vivo glucose bolus administration, the direction of the effect that we wanted to test was known, i.e. inhibition by CD3-403, hence a one tailed t-test was applied. All the statistical analyses were performed using GraphPad Prism 5 software.

STD-NMR. Cell membranes were prepared as described in Hoare et al. (132), with some modifications. Flp-In-293 cells (derived from the parent HEK293 cell line) stably transfected with ADRB2 and GNA15 expression constructs were grown in DMEM medium, containing penicillin (100 U/mL)-streptomycin (100 μg/mL) and fetal bovine serum (10%). Hygromycin (100 μg/mL) was added for the ADRB2 construct and puromycin (1 μg/mL) was added for the GNA15 construct as mentioned in the methods section for the calcium assay. Non-transfected HEK293 cells, which have a low endogenous expression of the β₂-AR, were grown in culture medium as described above, but without hygromycin and puromycin. After reaching confluence in a culture flask of 150 cm², monolayers of both cell lines were dislodged by trituration with their respective culture media. Cells were centrifuged at 150 g, 20° C. for 4 min, the supernatant medium was discarded, and the cells were washed with PBS. The cells were then re-suspended in 40 mL lysis buffer (25 mM TRIS, 2 mM EDTA, 6 mM MgCl₂ and 0.1 mM 4-(2-aminoethyl)benzenesulfonyl fluoride hydrochloride (AEBSF) pH 7.5, for 4 confluent flasks), kept at 4° C., and homogenized in an ice-cold glass Dounce homogenizer with 45 strokes. The homogenates were centrifuged at 1000 g, 4° C. for 10 min to remove intact cells. The supernatants were centrifuged at 40,000 g, 4° C. for 30 min. To ensure that the cell membrane preparations were guanine-nucleotide-free, the resulting pellet was washed with 30 mL lysis buffer, and finally suspended in buffer with 20 mM HEPES, 100 mM NaCl, 1 mM EDTA and 3 mM MgSO₄, pH 7.5. Total protein was quantified with the bicinchoninic acid assay (BCA assay) using bovine serum albumin (BSA) as the standard. Cell membranes were stored at −80° C. until use. Membranes were reconstituted in PBS for the STD-NMR assay.

NMR experiments were performed at 5° C. on a Bruker Avance II 600 NMR spectrometer equipped with a cryogenic TCI probe with a z-gradient. The standard Bruker pulse program stddiffesgp (54) was used for data collection using excitation sculpting to suppress the water signal and a 5-s STD saturation time. Data are collected with 32 k complex points for 2.5 s. A delay of 2 s is applied between each FID to ensure complete relaxation. For each of the STD experiments, 32 scans are accumulated. The spectra for both on-resonance and off-resonance saturation at resp. 0.7 and 12 ppm are collected interleaved. The Bruker command stdsplit was used to process and subtract on- and off-resonance FIDs.

Data availability. The authors declare that all data supporting the findings of this study are available within the article and its Supplementary Information files, and from the corresponding author upon reasonable request.

Study approval. All experiments with animals and isolated animal organs have been approved by the ethical commission of KU Leuven.

### Acknowledgements

We wish to thank S. Shirazi-Beechey (Liverpool) for the provision of RNA extracts from sheep mucosal scrapings and from STC-1 cells, and for stimulating discussions in the initiation phase of this work. We also thank L. Pardo and X. Deupi (Barcelona) for modeling work on the β₂-AR. We are also grateful to W. Van Driessche and A. S. Segal (Leuven) for help with the oocyte experiments and the use of the DSPOOC software, to J. Tytgat for kindly donating the pGEMHE vector, to J. Eggermont for provision of the M2 901/pBQ4.7 vector, to W. De Haes for help with the ANOVA statistical analysis, to B. Van Der Schueren for stimulating discussions, to J. Van der Heyden, Z. Nackaerts and L. Vanden Bosch for excellent technical assistance, to O. Van Den Bossche for help with the in vivo 4 g/kg glucose administration experiment and to N. Vangoethem for help with preparation of the figures. This work was supported by a predoctoral fellowship to FP from the Agency for Innovation by Science and Technology (IWT-Flanders), by mainly personal self-financing to WL and by grants from the COSAT program (Johnson & Johnson-VIB), the Fund for Scientific Research-Flanders, Interuniversity Attraction Poles Network P5/30 and P6/14, the Research Fund of the KU Leuven (Concerted Research Actions) and the Hercules Foundation (Flanders). The NMR study made use of the BioMacs facility at KU Leuven. Equipment in the facility was purchased with funds from the Flemish government (‘impuls project’) and FWO-Flanders.

### Author Contributions

F. P., C. P. K., E. L., S. D. G., A. M., P.iV. performed the experimental work; F. S., S. L., P. C., J. M., J. T., P. V. D., W. L., J. M. T. supervised the experimental work and contributed to the discussion of the results; F. S., W. L., J. M. T. designed the project; F. P., C. P. K., W. L., J. M. T. wrote the manuscript. F. P. initiated and performed the first part of the research, while C. P. K. carried out the second part of the work and completed the study. F. P. and C. P. K. were therefore designated in this order as co-first authors.

### Conflict of Interest

The authors declare possible competing interests. VIB/LRD has submitted a patent application on the use of the apical β-2-adrenergic receptor in enterocytes as a drug target (‘Role of the β-2-adrenergic receptor as sugar sensor in intestinal epithelial cells.’ 29 Aug. 2012. EP 12182118.5. Thevelein J. M., F. Paulussen, W. Luyten, P. Van Dijck).

### The Paper Explained

### Problem

Diabetes and obesity are two widespread diseases that could strongly profit from a precisely controlled reduction of sugar uptake from the gut. The sugar sensing systems that enhance sugar uptake from the gut could be appropriate drug targets but they are poorly understood. The glucose receptor system in the main intestinal epithelial cells, the enterocytes, has remained elusive up to now.

### Results

We show that the β₂-adrenergic receptor is strongly expressed at the apical membrane of the enterocytes, a highly unusual location for a hormone receptor of which the ligand, epinephrine, is distributed through the bloodstream. We show with several heterologous expression systems that the β₂-AR responds to different sugars in appropriate concentrations for a nutrient in the gut and that these responses are inhibited by well-known antagonists of β₂-AR. Epinephrine and sugars appear to bind to different but closely connected binding sites. With everted intestinal sacs of rats we show that epinephrine and sugars stimulate the uptake of glucose from the gut lumen compartment through SGLT1, the main glucose transporter in the enterocytes. This stimulation depends on β₂-AR and its PKA signaling pathway. Finally, using a novel non-bioavailable β₂-AR antagonist, we show that inhibition of β₂-AR in vivo results in a lowered increase in blood glucose levels after oral administration of a glucose bolus.

### Impact

The administration of SGLT1 or intestinal carbohydrase inhibitors to lower glucose uptake from the gut in diabetic and obese patients generally results in uncontrolled increases in free sugar levels in the gut causing osmotic diarrhea, enhanced microbial activity and flatulence. Complete inhibition of β₂-AR with a non-bioavailable antagonist has the advantage that only sugar-induced stimulation of sugar uptake by SGLT1 is blocked and not the total sugar uptake activity, strongly reducing the risk of unwanted side effects. Controlled lowering of post-prandial peaks in the blood glucose level in diabetic and obese patients is highly desirable for a beneficial medical treatment.

### REFERENCES

1. Dockray G J. Luminal sensing in the gut: an overview. J Physiol Pharmacol. 2003; 54 Suppl 4:9-17.
2. Ferraris R P. Dietary and developmental regulation of intestinal sugar transport. Biochem J. 2001; 360 (Pt 2): 265-76.
3. Diamond J M, and Karasov W H. Adaptive regulation of intestinal nutrient transporters. Proc Natl Acad Sci USA. 1987; 84 (8): 2242-5.
4. Dyer J, Vayro S, King T P, and Shirazi-Beechey S P. Glucose sensing in the intestinal epithelium. Eur J Biochem. 2003; 270 (16): 3377-88.
5. Shirazi-Beechey S P, Moran A W, Batchelor D J, Daly K, and Al-Rammahi M. Glucose sensing and signalling; regulation of intestinal glucose transport. Proc Nutr Soc. 2011; 70 (2): 185-93.
6. Sharp P A, Debnam E S, and Srai S K. Rapid enhancement of brush border glucose uptake after exposure of rat jejunal mucosa to. Gut. 1996; 39 (4): 545-50.
7. Miyamoto K, Hase K, Takagi T, Fujii T, Taketani Y, Minami H, et al. Differential responses of intestinal glucose transporter mRNA transcripts to levels of dietary sugars. Biochem J. 1993; 295 (Pt 1): 211-5.
8. Sharp P A, and Debnam E S. The role of cyclic AMP in the control of sugar transport across the brush-border and basolateral membranes of rat jejunal enterocytes. Exp Physiol. 1994; 79 (2): 203-14.
9. Dyer J, Salmon K S, Zibrik L, and Shirazi-Beechey S P. Expression of sweet taste receptors of the TIR family in the intestinal tract and enteroendocrine cells. Biochem Soc Trans. 2005; 33 (Pt 1): 302-5.
10. Margolskee R F, Dyer J, Kokrashvili Z, Salmon K S, Ilegems E, Daly K, et al. T1R3 and gustducin in gut sense sugars to regulate expression of Na+-glucose cotransporter 1. Proc Natl Acad Sci USA. 2007; 104 (38): 15075-80.
11. Buddington R K, Chen J W, and Diamond J M. Dietary regulation of intestinal brush-border sugar and amino acid transport in carnivores. Am J Physiol. 1991; 261 (4 Pt 2): R793-801.
12. Batchelor D J, Al-Rammahi M, Moran A W, Brand J G, Li X, Haskins M, et al. Sodium/glucose cotransporter-1, sweet receptor, and disaccharidase expression in the intestine of the domestic dog and cat: two species of different dietary habit. Am J Physiol Regul Integr Comp Physiol. 2011; 300 (1): R67-75.
13. Sternini C, Anselmi L, and Rozengurt E. Enteroendocrine cells: a site of ‘taste’ in gastrointestinal chemosensing. Curr Opin Endocrinol Diabetes Obes. 2008; 15 (1): 73-8.
14. Steinert R E, and Beglinger C. Nutrient sensing in the gut: interactions between chemosensory cells, visceral afferents and the secretion of satiation peptides. Physiol Behav. 2011; 105 (1): 62-70.
15. Glendinning J I. Oral Post-Oral Actions of Low-Calorie Sweeteners: A Tale of Contradictions and Controversies. Obesity (Silver Spring). 2018; 26 Suppl 3: S9-S17.
16. Ezcurra M, Reimann F, Gribble F M, and Emery E. Molecular mechanisms of incretin hormone secretion. Curr Opin Pharmacol. 2013; 13 (6): 922-7.
17. Kraakman L, Lemaire K, Ma P, Teunissen A W, Donaton M C, Van Dijck P, et al. A Saccharomyces cerevisiae G-protein coupled receptor, Gpr1, is specifically required for glucose activation of the CAMP pathway during the transition to growth on glucose. Mol Microbiol. 1999; 32 (5): 1002-12.
18. Lemaire K, Van de Velde S, Van Dijck P, and Thevelein J M. Glucose and sucrose act as agonist and mannose as antagonist ligands of the G protein-coupled receptor Gpr1 in the yeast Saccharomyces cerevisiae. Mol Cell. 2004; 16 (2): 293-9.
19. Welton R M, and Hoffman C S. Glucose monitoring in fission yeast via the Gpa2 galpha, the git5 Gbeta and the git3 putative glucose receptor. Genetics. 2000; 156 (2): 513-21.
20. Maidan M M, De Rop L, Serneels J, Exler S, Rupp S, Tournu H, et al. The G protein-coupled receptor Gpr1 and the Galpha protein Gpa2 act through the cAMP-protein kinase A pathway to induce morphogenesis in Candida albicans. Mol Biol Cell. 2005; 16 (4): 1971-86.
21. Miwa T, Takagi Y, Shinozaki M, Yun C W, Schell W A, Perfect J R, et al. Gpr1, a putative G-protein-coupled receptor, regulates morphogenesis and hypha formation in the pathogenic fungus Candida albicans. Eukaryot Cell. 2004; 3 (4): 919-31.
22. Li L, and Borkovich K A. GPR-4 is a predicted G-protein-coupled receptor required for carbon source-dependent asexual growth and development in Neurospora crassa. Eukaryot Cell. 2006; 5 (8): 1287-300.
23. Xue C, Hsueh Y P, and Heitman J. Magnificent seven: roles of G protein-coupled receptors in extracellular sensing in fungi. FEMS Microbiol Rev. 2008; 32 (6): 1010-32.
24. Rui L. Energy metabolism in the liver. Compr Physiol. 2014; 4 (1): 177-97.
25. Arner P. Human fat cell lipolysis: biochemistry, regulation and clinical role. Best Pract Res Clin Endocrinol Metab. 2005; 19 (4): 471-82.
26. Shukla A K, Sun J P, and Lefkowitz R J. Crystallizing thinking about the beta2-adrenergic receptor. Mol Pharmacol. 2008; 73 (5): 1333-8.
27. Frishman W H. beta-Adrenergic blockers: a 50-year historical perspective. Am J Ther. 2008; 15 (6): 565-76.
28. Cherezov V, Rosenbaum D M, Hanson M A, Rasmussen S G, Thian F S, Kobilka T S, et al. High-resolution crystal structure of an engineered human beta2-adrenergic G protein-coupled receptor. Science. 2007; 318 (5854): 1258-65.
29. Rasmussen S G, Choi H J, Rosenbaum D M, Kobilka T S, Thian F S, Edwards P C, et al. Crystal structure of the human beta2 adrenergic G-protein-coupled receptor. Nature. 2007; 450 (7168): 383-7.
30. Rosenbaum D M, Cherezov V, Hanson M A, Rasmussen S G, Thian F S, Kobilka T S, et al. GPCR engineering yields high-resolution structural insights into beta2-adrenergic receptor function. Science. 2007; 318 (5854): 1266-73.
31. Rasmussen S G, Choi H J, Fung J J, Pardon E, Casarosa P, Chae P S, et al. Structure of a nanobody-stabilized active state of the beta (2) adrenoceptor. Nature. 2011; 469 (7329): 175-80.
32. Rasmussen S G, DeVree B T, Zou Y, Kruse A C, Chung K Y, Kobilka T S, et al. Crystal structure of the beta2 adrenergic receptor-Gₛ protein complex. Nature. 2011; 477 (7366): 549-55.
33. Strosberg A D. Structure, function, and regulation of adrenergic receptors. Protein Sci. 1993; 2 (8): 1198-209.
34. Baker J G. The selectivity of beta-adrenoceptor antagonists at the human beta1, beta2 and beta3 adrenoceptors. Br J Pharmacol. 2005; 144 (3): 317-22.
35. Smith C, and Teitler M. Beta-blocker selectivity at cloned human beta 1- and beta 2-adrenergic receptors. Cardiovasc Drugs Ther. 1999; 13 (2): 123-6.
36. Tyndall J D, and Sandilya R. GPCR agonists and antagonists in the clinic. Med Chem. 2005; 1 (4): 405-21.
37. Vassilatis D K, Hohmann J G, Zeng H, Li F, Ranchalis J E, Mortrud M T, et al. The G protein-coupled receptor repertoires of human and mouse. Proc Natl Acad Sci USA. 2003; 100 (8): 4903-8.
38. Singh A K, Riederer B, Krabbenhoft A, Rausch B, Bonhagen J, Lehmann U, et al. Differential roles of NHERF1, NHERF2, and PDZK1 in regulating CFTR-mediated intestinal anion secretion in mice. J Clin Invest. 2009; 119 (3): 540-50.
39. Zhang J, Halm S T, and Halm D R. Adrenergic activation of electrogenic K+ secretion in guinea pig distal colonic epithelium: involvement of beta1- and beta2-adrenergic receptors. Am J Physiol Gastrointest Liver Physiol. 2009; 297 (2): G269-77.
40. Olsen W A, and Ingelfinger F J. The role of sodium in intestinal glucose absorption in man. J Clin Invest. 1968; 47 (5): 1133-42.
41. Ferraris R P, Yasharpour S, Lloyd K C, Mirzayan R, and Diamond J M. Luminal glucose concentrations in the gut under normal conditions. Am J Physiol. 1990; 259 (5 Pt 1): G822-37.
42. Minuth M, and Jakobs K H. Sodium regulation of agonist and antagonist binding to beta-adrenoceptors in intact and Ns-deficient membranes. Naunyn Schmiedebergs Arch Pharmacol. 1986; 333 (2): 124-9.
43. Bokoch M P, Zou Y, Rasmussen S G, Liu C W, Nygaard R, Rosenbaum D M, et al. Ligand-specific regulation of the extracellular surface of a G-protein-coupled receptor. Nature. 2010; 463 (7277): 108-12.
44. Canals M, Sexton P M, and Christopoulos A. Allostery in GPCRs: ‘MWC’ revisited. Trends Biochem Sci. 2011; 36 (12): 663-72.
45. Alam M A, Al-Jenoobi F I, and Al-Mohizea A M. Everted gut sac model as a tool in pharmaceutical research: limitations and applications. J Pharm Pharmacol. 2012; 64 (3): 326-36.
46. Hamilton K L, and Butt A G. Glucose transport into everted sacs of the small intestine of mice. Adv Physiol Educ. 2013; 37 (4): 415-26.
47. Yu L C, Huang C Y, Kuo W T, Sayer H, Turner J R, and Buret A G. SGLT-1-mediated glucose uptake protects human intestinal epithelial cells against Giardia duodenalis-induced apoptosis. Int J Parasitol. 2008; 38 (8-9): 923-34.
48. Nalli A D, Kumar D P, Mahavadi S, Al-Shboul O, Alkahtani R, Kuemmerle J F, et al. Hypercontractility of intestinal longitudinal smooth muscle induced by cytokines is mediated by the nuclear factor-kappaB/AMP-activated kinase/myosin light chain kinase pathway. J Pharmacol Exp Ther. 2014; 350 (1): 89-98.
49. Tazawa S, Yamato T, Fujikura H, Hiratochi M, Itoh F, Tomae M, et al. SLC5A9/SGLT4, a new Na+-dependent glucose transporter, is an essential transporter for mannose, 1,5-anhydro-D-glucitol, and fructose. Life Sci. 2005; 76 (9): 1039-50.
50. Wright E M, Loo D D, and Hirayama B A. Biology of human sodium glucose transporters. Physiol Rev. 2011; 91 (2): 733-94.
51. Assadi-Porter F M, Tonelli M, Maillet E, Hallenga K, Benard O, Max M, et al. Direct NMR detection of the binding of functional ligands to the human sweet receptor, a heterodimeric family 3 GPCR. J Am Chem Soc. 2008; 130 (23): 7212-3.
52. Assadi-Porter F M, Tonelli M, Maillet E L, Markley J L, and Max M. Interactions between the human sweet-sensing TIR2-T1R3 receptor and sweeteners detected by saturation transfer difference NMR spectroscopy. Biochim Biophys Acta. 2010; 1798 (2): 82-6.
53. Venkitakrishnan R P, Benard O, Max M, Markley J L, and Assadi-Porter F M. Use of NMR saturation transfer difference spectroscopy to study ligand binding to membrane proteins. Methods Mol Biol. 2012; 914:47-63.
54. Mayer M, and Meyer B. Characterization of ligand binding by Saturation Transfer Difference NMR Spectroscopy. Angew Chem Int Ed Engl. 1999; 38 (12): 1784-8.
55. Boivin V, Jahns R, Gambaryan S, Ness W, Boege F, and Lohse M J. Immunofluorescent imaging of beta 1- and beta 2-adrenergic receptors in rat kidney. Kidney Int. 2001; 59 (2): 515-31.
56. Blake W D. Effect of epinephrine on tubular reabsorption of glucose by dog kidney. Am J Physiol. 1962; 202:897-900.
57. Goff J P. Invited review: Mineral absorption mechanisms, mineral interactions that affect acid-base and antioxidant status, and diet considerations to improve mineral status. J Dairy Sci. 2018; 101 (4): 2763-813.
58. Yu O, and Ouyang A. Distribution of beta-adrenoceptor subtypes in gastrointestinal tract of nondiabetic and diabetic BB rats. A longitudinal study. Dig Dis Sci. 1997; 42 (6): 1146-53.
59. Vijftigschild L A, Berkers G, Dekkers J F, Zomer-van Ommen D D, Matthes E, Kruisselbrink E, et al. beta2-Adrenergic receptor agonists activate CFTR in intestinal organoids and subjects with cystic fibrosis. Eur Respir J. 2016; 48 (3): 768-79.
60. Mailleau C, Capeau J, and Brahimi-Horn M C. Interrelationship between the Na+/glucose cotransporter and CFTR in Caco-2 cells: relevance to cystic fibrosis. J Cell Physiol. 1998; 176 (3): 472-81.
61. Re G, Badino P, De Angelis I, Odore R, Belloli C, Stammati A, et al. Identification and coupling to adenylate cyclase of three different [(3) H]CGP 12177 binding sites in Caco-2 cell membranes. Pharmacol Res. 2001; 43 (4): 393-8.
62. Wright E M, Hirsch J R, Loo D D, and Zampighi G A. Regulation of Na+/glucose cotransporters. J Exp Biol. 1997; 200 (Pt 2): 287-93.
63. Vestri S, Okamoto M M, de Freitas H S, Aparecida Dos Santos R, Nunes M T, Morimatsu M, et al. Changes in sodium or glucose filtration rate modulate expression of glucose transporters in renal proximal tubular cells of rat. J Membr Biol. 2001; 182 (2): 105-12.
64. Shepard B D, and Pluznick J L. Saving the sweetness: renal glucose handling in health and disease. Am J Physiol Renal Physiol. 2017; 313 (1): F55-F61.
65. Subramanian S, Glitz P, Kipp H, Kinne R K, and Castaneda F. Protein kinase-A affects sorting and conformation of the sodium-dependent glucose co-transporter SGLT1. J Cell Biochem. 2009; 106 (3): 444-52.
66. Schafer N, Rikkala P R, Veyhl-Wichmann M, Keller T, Jurowich C F, Geiger D, et al. A Modified Tripeptide Motif of RS1 (RSC1A1) Down-Regulates Exocytotic Pathways of Human Na (+)-d-glucose Cotransporters SGLT1, SGLT2, and Glucose Sensor SGLT3 in the Presence of Glucose. Mol Pharmacol. 2019; 95 (1): 82-96.
67. Doenst T, and Taegtmeyer H. alpha-adrenergic stimulation mediates glucose uptake through phosphatidylinositol 3-kinase in rat heart. Circ Res. 1999; 84 (4): 467-74.
68. Faintrenie G, and Geloen A. Alpha-1 adrenergic stimulation of glucose uptake in rat white adipocytes. J Pharmacol Exp Ther. 1998; 286 (2): 607-10.
69. Abe H, Minokoshi Y, and Shimazu T. Effect of a beta 3-adrenergic agonist, BRL35135A, on glucose uptake in rat skeletal muscle in vivo and in vitro. J Endocrinol. 1993; 139 (3): 479-86.
70. Chernogubova E, Cannon B, and Bengtsson T. Norepinephrine increases glucose transport in brown adipocytes via beta3-adrenoceptors through a cAMP, PKA, and PI3-kinase-dependent pathway stimulating conventional and novel PKCs. Endocrinology. 2004; 145 (1): 269-80.
71. Catus SL, Gibbs M E, Sato M, Summers R J, and Hutchinson D S. Role of beta-adrenoceptors in glucose uptake in astrocytes using beta-adrenoceptor knockout mice. Br J Pharmacol. 2011; 162 (8): 1700-15.
72. Hutchinson D S, Chernogubova E, Dallner O S, Cannon B, and Bengtsson T. Beta-adrenoceptors, but not alpha-adrenoceptors, stimulate AMP-activated protein kinase in brown adipocytes independently of uncoupling protein-1. Diabetologia. 2005; 48 (11): 2386-95.
73. Nevzorova J, Bengtsson T, Evans B A, and Summers R J. Characterization of the beta-adrenoceptor subtype involved in mediation of glucose transport in L6 cells. Br J Pharmacol. 2002; 137 (1): 9-18.
74. Kanda Y, and Watanabe Y. Adrenaline increases glucose transport via a Rap1-p38MAPK pathway in rat vascular smooth muscle cells. Br J Pharmacol. 2007; 151 (4): 476-82.
75. Ngala R A, O'Dowd J, Wang S J, Agarwal A, Stocker C, Cawthorne M A, et al. Metabolic responses to BRL37344 and clenbuterol in soleus muscle and C2C12 cells via different atypical pharmacologies and beta2-adrenoceptor mechanisms. Br J Pharmacol. 2008; 155 (3): 395-406.
76. Nevzorova J, Evans B A, Bengtsson T, and Summers R J. Multiple signalling pathways involved in beta2-adrenoceptor-mediated glucose uptake in rat skeletal muscle cells. Br J Pharmacol. 2006; 147 (4): 446-54.
77. Liu Y L, and Stock M J. Acute effects of the beta 3-adrenoceptor agonist, BRL 35135, on tissue glucose utilisation. Br J Pharmacol. 1995; 114 (4): 888-94.
78. Dong J H, Chen X, Cui M, Yu X, Pang Q, and Sun J P. beta2-adrenergic receptor and astrocyte glucose metabolism. J Mol Neurosci. 2012; 48 (2): 456-63.
79. Dehvari N, Hutchinson D S, Nevzorova J, Dallner O S, Sato M, Kocan M, et al. beta (2)-Adrenoceptors increase translocation of GLUT4 via GPCR kinase sites in the receptor C-terminal tail. Br J Pharmacol. 2012; 165 (5): 1442-56.

80. Mukaida S, Sato M, Oberg A I, Dehvari N, Olsen J M, Kocan M, et al. BRL37344 stimulates GLUT4 translocation and glucose uptake in skeletal muscle via beta2-adrenoceptors without causing classical receptor desensitization. Am J Physiol Regul Integr Comp Physiol. 2019; 316 (5): R666-R77.
81. Shirazi-Beechey S P, Moran A W, Bravo D, and Al-Rammahi M. Nonruminant Nutrition Symposium: intestinal glucose sensing and regulation of glucose absorption: implications for swine nutrition. J Anim Sci. 2011; 89 (6): 1854-62.
82. Stearns A T, Balakrishnan A, Rhoads D B, and Tavakkolizadeh A. Rapid upregulation of sodium-glucose transporter SGLT1 in response to intestinal sweet taste stimulation. Ann Surg. 2010; 251 (5): 865-71.
83. Gromova L V, Fetissov S O, and Gruzdkov A A. Mechanisms of Glucose Absorption in the Small Intestine in Health and Metabolic Diseases and Their Role in Appetite Regulation. Nutrients. 2021; 13 (7).
84. Thevelein J M, and Voordeckers K. Functioning and evolutionary significance of nutrient transceptors. Mol Biol Evol. 2009; 26 (11): 2407-14.
85. Nordstrom K J, Sallman Almen M, Edstam M M, Fredriksson R, and Schioth H B. Independent HHsearch, Needleman—Wunsch-based, and motif analyses reveal the overall hierarchy for most of the G protein-coupled receptor families. Mol Biol Evol. 2011; 28 (9): 2471-80.
86. de Mendoza A, Sebe-Pedros A, and Ruiz-Trillo I. The evolution of the GPCR signaling system in eukaryotes: modularity, conservation, and the transition to metazoan multicellularity. Genome Biol Evol. 2014; 6 (3): 606-19.
87. Krishnan A, Almen M S, Fredriksson R, and Schioth H B. The origin of GPCRs: identification of mammalian like Rhodopsin, Adhesion, Glutamate and Frizzled GPCRs in fungi. PLoS One. 2012; 7 (1): e29817.
88. Graul R C, and Sadee W. Evolutionary relationships among G protein-coupled receptors using a clustered database approach. AAPS PharmSci. 2001; 3 (2): E12.
89. Wiejak J, Surmacz L, and Wyroba E. Immunoanalogue of vertebrate beta-adrenergic receptor in the unicellular eukaryote Paramecium. Histochem J. 2002; 34 (1-2): 51-6.
90. Wiejak J, Surmacz L, and Wyroba E. Dynamin-association with agonist-mediated sequestration of beta-adrenergic receptor in single-cell eukaryote Paramecium. J Exp Biol. 2004; 207 (Pt 10): 1625-32.
91. Platek A, Wiejak J, and Wyroba E. RT-PCR and Northern blot analysis in search for a putative Paramecium beta-adrenergic receptor. Acta Biochim Pol. 1999; 46 (3): 813-21.
92. Wyroba E. Stimulation of Paramecium phagocytosis by phorbol ester and forskolin. Cell Biol Int Rep. 1987; 11 (9): 657-64.
93. Wyroba E. Beta-adrenergic stimulation of phagocytosis in the unicellular eukaryote Paramecium aurelia. Cell Biol Int Rep. 1989; 13 (8): 667-78.
94. de Castro S L, and Oliveira M M. Radioligand binding characterization of beta-adrenergic receptors in the protozoa Trypanosoma cruzi. Comp Biochem Physiol C. 1987; 87 (1): 5-8.
95. Ghanouni P, Steenhuis J J, Farrens D L, and Kobilka B K. Agonist-induced conformational changes in the G-protein-coupling domain of the beta 2 adrenergic receptor. Proc Natl Acad Sci USA. 2001; 98 (11): 5997-6002.
96. Thevelein J M, and de Winde J H. Novel sensing mechanisms and targets for the CAMP-protein kinase A pathway in the yeast Saccharomyces cerevisiae. Mol Microbiol. 1999; 33 (5): 904-18.
97. Aulsebrook K A. Intestinal absorption of glucose and sodium: Effects of epinephrine and norepinephrine. Biochem Biophys Res Commun. 1965; 18:165-9.
98. Ishikawa Y, Eguchi T, and Ishida H. Mechanism of beta-adrenergic agonist-induced transmural transport of glucose in rat small intestine. Regulation of phosphorylation of SGLT1 controls the function. Biochim Biophys Acta. 1997; 1357 (3): 306-18.
99. Bochain A, Estey L, Haronian G, Reale M, Rojas C, and Cramer J. Determination of catecholamine permeability coefficients for passive diffusion across phospholipid vesicle membranes. J Membr Biol. 1981; 60 (1): 73-6.
100. Aschenbach J R, Borau T, and Gabel G. Glucose uptake via SGLT-1 is stimulated by beta (2)-adrenoceptors in the ruminal epithelium of sheep. J Nutr. 2002; 132 (6): 1254-7.
101. Taatjes D J, and Roth J. Glycosylation in intestinal epithelium. Int Rev Cytol. 1991; 126:135-93.
102. Biol M C, Martin A, and Louisot P. Nutritional and developmental regulation of glycosylation processes in digestive organs. Biochimie. 1992; 74 (1): 13-24.
103. Moremen K W, Tiemeyer M, and Nairn A V. Vertebrate protein glycosylation: diversity, synthesis and function. Nat Rev Mol Cell Biol. 2012; 13 (7): 448-62.
104. Asano T, Ogihara T, Katagiri H, Sakoda H, Ono H, Fujishiro M, et al. Glucose transporter and Na+/glucose cotransporter as molecular targets of anti-diabetic drugs. Curr Med Chem. 2004; 11 (20): 2717-24.
105. Wagman A S, and Nuss J M. Current therapies and emerging targets for the treatment of diabetes. Curr Pharm Des. 2001; 7 (6): 417-50.
106. Osswald C, Baumgarten K, Stumpel F, Gorboulev V, Akimjanova M, Knobeloch K P, et al. Mice without the regulator gene Rsc1A1 exhibit increased Na+-D-glucose cotransport in small intestine and develop obesity. Mol Cell Biol. 2005; 25 (1): 78-87.
107. Dyer J, Wood I S, Palejwala A, Ellis A, and Shirazi-Beechey S P. Expression of monosaccharide transporters in intestine of diabetic humans. Am J Physiol Gastrointest Liver Physiol. 2002; 282 (2): G241-8.
108. Koepsell H. Glucose transporters in the small intestine in health and disease. Pflugers Arch. 2020; 472 (9): 1207-48.
109. Nguyen N Q, Debreceni T L, Bambrick J E, Chia B, Wishart J, Deane A M, et al. Accelerated intestinal glucose absorption in morbidly obese humans: relationship to glucose transporters, incretin hormones, and glycemia. J Clin Endocrinol Metab. 2015; 100 (3): 968-76.
110. Soergel K H. Colonic fermentation: metabolic and clinical implications. Clin Investig. 1994; 72 (10): 742-8.
111. Wright E M, Hirayama B A, and Loo D F. Active sugar transport in health and disease. J Intern Med. 2007; 261 (1): 32-43.
112. Santulli G, Lombardi A, Sorriento D, Anastasio A, Del Giudice C, Formisano P, et al. Age-related impairment in insulin release: the essential role of beta (2)-adrenergic receptor. Diabetes. 2012; 61 (3): 692-701.
113. Skikama H, and Ui M. Adrenergic receptor and epinephrine-induced hyperglycemia and glucose tolerance. Am J Physiol. 1975; 229 (4): 962-6.
114. Holm G, Johansson S, Vedin A, Wilhelmsson C, and Smith U. The effect of beta-blockade on glucose tolerance and insulin release in adult diabetes. Acta Med Scand. 1980; 208 (3): 187-91.
115. Moore C A, Milano S K, and Benovic J L. Regulation of receptor trafficking by GRKs and arrestins. Annu Rev Physiol. 2007; 69:451-82.
116. Yudowski G A, Puthenveedu M A, Henry A G, and von Zastrow M. Cargo-mediated regulation of a rapid Rab4-dependent recycling pathway. Mol Biol Cell. 2009; 20 (11): 2774-84.
117. Barron A J, Zaman N, Cole G D, Wensel R, Okonko D O, and Francis D P. Systematic review of genuine versus spurious side-effects of beta-blockers in heart failure using placebo control: recommendations for patient information. Int J Cardiol. 2013; 168 (4): 3572-9.
118. Sears M R. Adverse effects of beta-agonists. J Allergy Clin Immunol. 2002; 110 (6 Suppl): S322-8.
119. Leonetti G, and Egan C G. Use of carvedilol in hypertension: an update. Vasc Health Risk Manag. 2012; 8:307-22.
120. Messerli F H, and Grossman E. beta-Blockers in hypertension: is carvedilol different? Am J Cardiol. 2004; 93 (9A): 7B-12B.
121. Nilsson P M, Cederholm J, Zethelius B R, Eliasson B R, Eeg-Olofsson K, and Gudbj Rnsdottir S. Trends in blood pressure control in patients with type 2 diabetes: data from the Swedish National Diabetes Register (NDR). Blood Press. 2011; 20 (6): 348-54.
122. Elayan H, Milic M, Sun P, Gharaibeh M, and Ziegler M G. Chronic beta2 adrenergic agonist, but not exercise, improves glucose handling in older type 2 diabetic mice. Cell Mol Neurobiol. 2012; 32 (5): 871-7.
123. Ziegler M G, Elayan H, Milic M, Sun P, and Gharaibeh M. Epinephrine and the metabolic syndrome. Curr Hypertens Rep. 2012; 14 (1): 1-7.
124. Jensen J, Ruzzin J, Jebens E, Brennesvik E O, and Knardahl S. Improved insulin-stimulated glucose uptake and glycogen synthase activation in rat skeletal muscles after adrenaline infusion: role of glycogen content and PKB phosphorylation. Acta Physiol Scand. 2005; 184 (2): 121-30.
125. Ernande L, Stanford K I, Thoonen R, Zhang H, Clerte M, Hirshman M F, et al. Relationship of brown adipose tissue perfusion and function: a study through beta2-adrenoreceptor stimulation. J Appl Physiol (1985). 2016; 120 (8): 825-32.
126. Cole S W, and Sood A K. Molecular pathways: beta-adrenergic signaling in cancer. Clin Cancer Res. 2012; 18 (5): 1201-6.
127. Perez-Sayans M, Somoza-Martin J M, Barros-Angueira F, Gayoso-Diz P, Otero-Rey E M, Gandra-Rey J M, et al. Activity of beta2-adrenergic receptor in oral squamous cell carcinoma is mediated by overexpression of the ADRBK2 gene: a pilot study. Biotech Histochem. 2012; 87 (3): 179-86.
128. Ji Y, Chen S, Xiao X, Zheng S, and Li K. beta-blockers: a novel class of antitumor agents. Onco Targets Ther. 2012; 5:391-401.
129. Chakir K, Xiang Y, Yang D, Zhang S J, Cheng H, Kobilka B K, et al. The third intracellular loop and the carboxyl terminus of beta2-adrenergic receptor confer spontaneous activity of the receptor. Mol Pharmacol. 2003; 64 (5): 1048-58.
130. Margulis L, Banerjee S, and White T. Colchicine-inhibited cilia regeneration: explanation for lack of effect in tris buffer medium. Science. 1969; 164 (3884): 1177-8.
131. Raba J, and Mottola H A. Glucose oxidase as an analytical reagent. Crit Rev Anal Chem. 1995; 25:1-42.
132. Hoare S R, and Usdin T B. Quantitative cell membrane-based radioligand binding assays for parathyroid hormone receptors. J Pharmacol Toxicol Methods. 1999; 41 (2-3): 83-90.

### Supplementary Information Figure Legends

### Supplementary FIG. 1

Effect of 1 μM epinephrine (Epi) and 1 μM epinephrine (Epi) together with 1 μM ICI 118,551 (ICI), a β₂-AR-specific inhibitor, on calcium response in HEK-293 cells. Epinephrine did not elicit a calcium response in HEK-293 cells via the endogenous β₂-AR. X-axis shows time in s, and y-axis displays fluorescence intensity (arbitrary units) (n=3). All values are expressed as mean±SEM.

### Supplementary FIG. 2

Effect of 1 μM ICI-118,551 (ICI), a β₂-AR-specific antagonist, in Flp-In-293 cells stably transfected with ADRB2 and GNA15. ICI-118,551 itself did not elicit a calcium response. Epinephrine (5 nM) was added as positive control, and Hank's Buffered Salt Solution (HBSS) as negative control. X-axis shows time in s and y-axis displays fluorescence intensity (arbitrary units) (n=3). All values are expressed as mean±SEM.

### Supplementary FIG. 3

Absence of maltose response in Xenopus oocytes lacking heterologously expressed human β₂-AR. Forskolin/IBMX, which artificially increases the CAMP level, was added to demonstrate functionality of the CFTR channel.

### Supplementary FIG. 4

Binding (mean±SEM) of ¹²⁵I-cyanopindolol to vesicles containing β₂-AR. a, Stimulation of the binding with increasing concentrations of glucose (in the absence of Na⁺), b, Stimulation of the binding with increasing concentrations of Na⁺ (in the absence of glucose). Note that the Y-axis starts at 50%.

### Supplementary FIG. 5

Inhibition of epinephrine and sugar responses with β₂-AR-specific antagonists. a, 200 μM propranolol, b, 200 μM labetalol, c, 200 μM nadolol. 20 nM epinephrine or 70 mM sugar was added in the absence (●) or presence of inhibitor (◯).

### Supplementary FIG. 6

Differential inhibition of the sugar response with a β1-antagonist, metoprolol (200 μM); 20 nM epinephrine or 70 mM sugar was added in the absence (●) or presence (◯) of antagonist.

### Supplementary FIG. 7

Absence of inhibition of the epinephrine response and differential inhibition of the sugar response with β1-antagonists. a, 200 μM acebutolol, b, 200 μM atenolol. 20 nM epinephrine or 70 mM sugar was added in the absence (●) or presence (◯) of antagonist.

### Supplementary FIG. 8

Effect of phlorizin (PZ) (100 μM) and LX4211 (2 μM) on glucose (Glu) accumulated after 75 min inside everted sacs, prepared from rat intestine. PZ and LX4211 inhibited more than 90% of total glucose transport. (Respective number of replicates n=2, n=2, n=2 for conditions shown in the figure. For conditions with multiple measurements, the low and high values are indicated as the top and bottom of the box, with a horizontal line at the mean.)

### Supplementary FIG. 9

Effect of ICI-118,551 (ICI) and phlorizin (PZ) on glucose (Glu) accumulated after 10 min inside everted sacs, prepared from rat intestine. ICI itself did not have any effect on glucose transport. (Respective number of replicates n=6, n=7, n=3 for conditions shown in the figure; LLOQ is lower limit of quantification). The glucose transport for the different groups was expressed as a % of the glucose transport of the glucose control group. All values are expressed as mean±SEM, p>0.05: ns.

### Supplementary Table 1

GPCRs whose expression was detected in mouse proximal duodenal scrapings or in the intestinal STC-1 cell line cultured in medium with 5 or 25 mM glucose for the last 24 h.

### Supplementary Table 2

Caco-2 cell permeability data of compound CD3-403 (apical to basal (A-B) and basal to apical (B-A), measured at pH 7.4 in both apical and basal compartment)

### Supplementary Table 3

### Pcr Primers

SUPPLEMENTARY TABLE 1
GPCRs whose expression was detected in mouse proximal duodenal
scrapings or in the intestinal STC-1 cell line cultured
in medium with 5 or 25 mM glucose for the last 24 h.

| | PCR | | | Micro-Array | | qPCR | |
|---|---|---|---|---|---|---|---|
| | | STC-1 | | STC-1 | | STC-1 | |
| | | 5 | 25 | 5 | 25 | 5 | 25 |
| | | mM | mM | mM | mM | mM | mM |
| | mouse | Glu | Glu | Glu | Glu | Glu | Glu |
| 5-HT1b | | | | + | + | | |
| 5-HT3a | | | | + | + | | |
| ADRB2 | + | + | | / | / | | |
| AGTRL1 | + | | + | − | − | + | + |
| ATIIR | | | | − | + | | |
| celsr3 | | | | + | + | | |
| GPR1 | + | − | | + | + | | + |
| GPR120 | + | − | | + | + | | + |
| GPR125 | + | + | | / | / | | + |
| GPR151 | + | | + | / | / | + | + |
| GPR18 | − | − | | + | + | | + |
| GPR19 | + | + | | + | + | | + |
| GPR22 | − | − | | / | / | | + |
| GPR35 | + | | + | / | / | + | + |
| GPR39 | + | − | | + | + | | + |
| GPR40 | + | + | | / | / | | + |
| GPR43 | + | + | | + | + | | |
| GPR48 | + | + | | + | + | | + |
| GPR49 | − | + | | + | + | | + |
| GPR55 | − | | − | / | / | | |
| GPR56 | + | + | | + | + | | + |
| GPR61 | + | − | | + | + | | + |
| GPR73 | − | | − | − | − | | |
| GPR80 | + | − | | / | / | | + |
| GPR85 | + | + | | + | + | | + |
| GPR88 | − | − | | + | + | | + |
| GPRC5C | + | | + | + | + | + | + |
| PGR22 | + | | + | / | / | | |
| prosta E4 | | | | + | + | | |
| RDC1 | + | | + | − | − | + | + |
| TEM5 | − | | + | − | − | + | + |
| TM7SF1 | + | − | | + | + | | + |
| TM7SF2 | + | + | | + | + | | + |
| TM7SF3 | + | + | | + | + | | + |
| TPRA40 | + | | + | + | + | + | + |

+, positive detection;
−, no detection;
/, not present on micro-array;
Glu, glucose

SUPPLEMENTARY TABLE 2
Caco-2 cell permeability data of compound CD3-403 (apical to basal (A-B) and basal
to apical (B-A), measured at pH 7.4 in both apical and basal compartment)

| | | A-B | Mean | B-A | Mean | |
|---|---|---|---|---|---|---|
| | Test | permeability | Recovery | permeability | Recovery | |
| Compound | Concentration | (10⁻⁶ cm/s) | (%) | (10⁻⁶ cm/s) | (%) | Efflux |
| CD3-403 | 1.0 μM | 0.4 | 83 | 1.3 | 94 | 3.25 |

Low permeability: <2 × 10⁻⁶ cm/s
Medium permeability: 2 × 10⁻⁶ cm/s < permeability < 20 × 10⁻⁶ cm/s)
High permeability: >20 × 10⁻⁶ cm/s

Supplementary Table 3
PCR primers
Forward (fw) and reverse (re) primers used for
reverse transcriptase PCR and for quantitative
PCR (qPCR) were designed as required for SYBR
Green (Eurogentec) qPCR reactions.

GPR1 fw TGGACCCCTTATCACCTGTTTAG
GPR1 re CCTGCAGCACATTCTGGAAA
GPR120 fw TCCGAGTGTCCCAACAAGACTA
GPR120 re ATGATGGGACTCCACATGATG
GPR125 fw CGTGCAGTTTCGAACAAACG
GPR125 re CTGGCCCGGTGTCCTTT
GPR18 fw GGAGAAGTCCATACGGATCATCA
GPR18 re GTGGAAGGGCACGAAGCA
GPR19 fw CTACCGCAGCAATGCCTACA
GPR19 re GATTTCCGAAATGCCCACAT
GPR22 fw TCGTGTTTGGTGTGAGAACTTCA
GPR22 re TCCCGGTGGCGTTTCA
GPR39 fw CCCATGGAGTTCTACAGCATCAT
GPR39 re CGTGTGGAGCTTACAGGACAGA
GPR40 fw TGCCTCCAATGTGGCTAGTTT
GPR40 re CCCTGTGATGAGTCCCAACTTC
GPR48 fw AAGACGACTGGAAGCTCCTGAA
GPR48 re CCGCCTTGGCTGCTGAT
GPR49 fw TGAGATCAAAACACGCGAGTCT
GPR49 re TGCTGGCGTGGGTAAAGG
GPR56 fw CCAAGGCTTCCTCATCTTCCT
GPR56 re GCGCTGTCTGAGTTGTTCTTCA
GPR61 fw AACCTGGATTGGCTACTTTTGC
GPR61 re TAAGCTCGCCCCGGATCT
GPR80 fw CCTTTGGCAACCTGCTGTTATAT
GPR80 re CCGCTGGCTTTGCATCTC
GPR85 fw TTGCACGAGGGCCTGTAGTAC
GPR85 re ATTCCTGCTTGGGCGAAACT
GPR88 fw GCTCTACACGTGGAGGAACGA
GPR88 re GTTGCGCCTGGGACATG
TM7SF1 fw CCCCCGAAGATATGACAGTGAT
TM7SF1 re GGAGCAAAACTTCCCTGAAGTC
TM7SF2 fw GCAGCAGTGCCTCCAAAAGT
TM7SF2 re GGTAAGGCACACGCTTGCA
TM7SF3 fw GAAAGAGGACAGCCGCCTTT
TM7SF3 re GATGTTGGTCACTCTGCGTTCTC

### Structural Activity Relationship (SAR) for CD3 Compounds

### Protocol

Cell line: HEK293 cells stably transfected with ß2AR
Epinephrine concentration: 5 nM
Number of cells/well: 40,000
Instrument: Flexstation II (Molecular Devices)
Fluo-4 NW Calcium Assay Kit (Molecular Probes-Invitrogen)
Inhibitor pre-incubation time: 45 min
Data analysis: GraphPad Prism 5

CD3 compounds overview

| Conc. (mM) | % INHIBTION | IC₅₀ (microM) | Ki (microM) |
|---|---|---|---|
| A | 20 | . | .. |
| B | 100 | . | .. |
| C | 67 | 18.5 | 0.003719 |
| D | 61 | . | . |
| E (Precipitated) | 96 | 0.5 | 15-04 μM |
| F | 78 | . | . |
| G | 100 | 0.71 | 0.000142 |
| H | 96 | 2.3 | 0.00046 |
| I | −2 | . | .. |
| J | 0 | . | . |
| K | 0 | . | . |
| L | 12 | . | . |
| M | 51 | 28 | 0.006599 |
| N | 4 | . | . |
| O | 48 | . | . |
| P | −11 | . | .. |
| S | 5 | . | .. |

Concentration used for inhibitors: 33.33 microM
Note:
Numbers in red colour indicates toxicity to cells
${K}_{i} = \frac{{IC}_{50}}{\frac{[ A ]}{{EC}_{50}} + 1}$ Ki: Inhibition constant
IC₅₀: Half maximal inhibitory concentration
[A]: Fixed concentration of agonist
EC₅₀: Half maximal activation of receptor (0.001 nM)

CD3 compounds overview

| Conc. (nM) | % INHIBITION | IC50 (microM) | Ki (microM) |
|---|---|---|---|
| T | 97 | 0.04 | [symbol] |
| U | 72 | 44 | [symbol] |
| W | 48 | — | — |
| X | 95 | 0.07 | 1.4E−05 |
| Y | −4 | — | — |
| Z | 97 | 0.02 | [symbol] |
| ALPHA | 79 | [symbol] | [symbol] |
| BETA | 36 | — | — |
| GAMMA | −3 | — | — |
| ICI-118551 | — | 0.004 | [symbol] |
| Butoxamine | — | 14.3 | [symbol] |

[symbol]

indicates data missing or illegible when filed

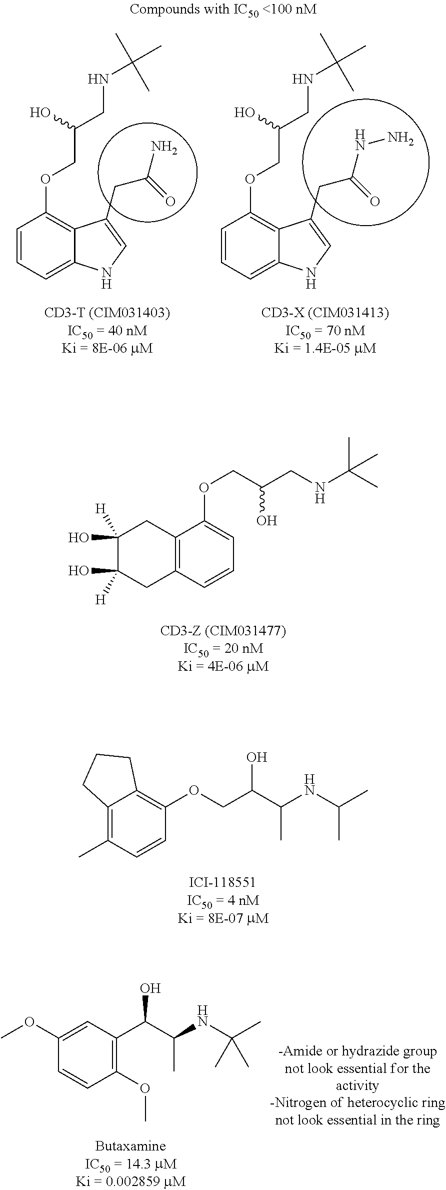

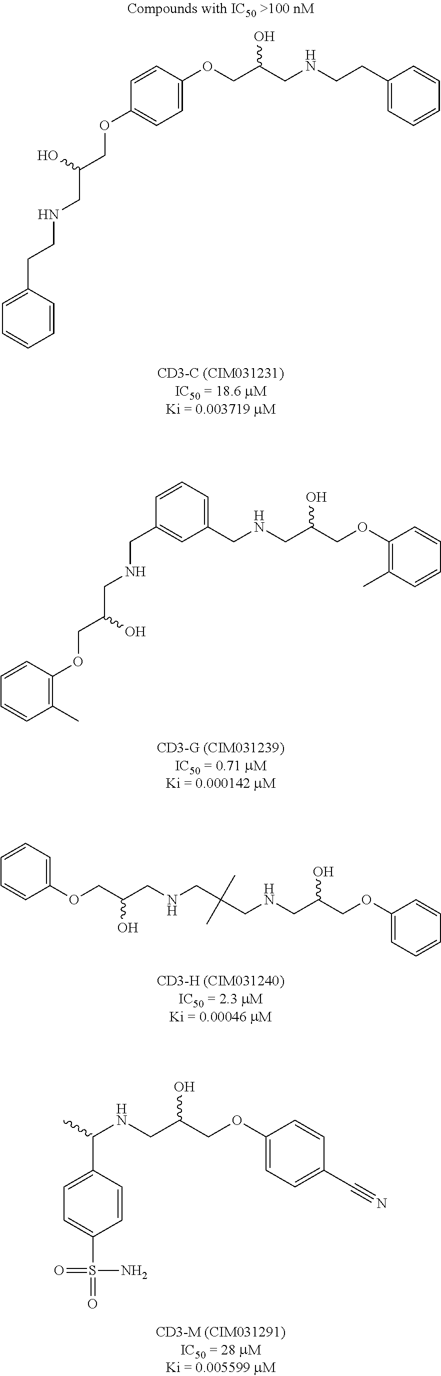 [SMILES: OC(CNCCc1ccccc1)COc1ccc(OCC(O)CNCCc2ccccc2)cc1 ; Cc1ccccc1OCC(O)CNCc1cccc(CNCC(O)COc2ccccc2C)c1 ; CC(C)(CNCC(O)COc1ccccc1)CNCC(O)COc1ccccc1 ; CC(NCC(O)COc1ccc(C#N)cc1)c1ccc(S(N)(=O)=O)cc1]

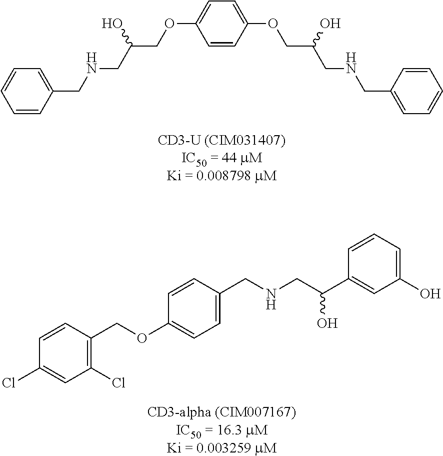 [SMILES: OC(CNCc1ccccc1)COc1ccc(OCC(O)CNCc2ccccc2)cc1 ; Oc1cccc(C(O)CNCc2ccc(OCc3ccc(Cl)cc3Cl)cc2)c1]

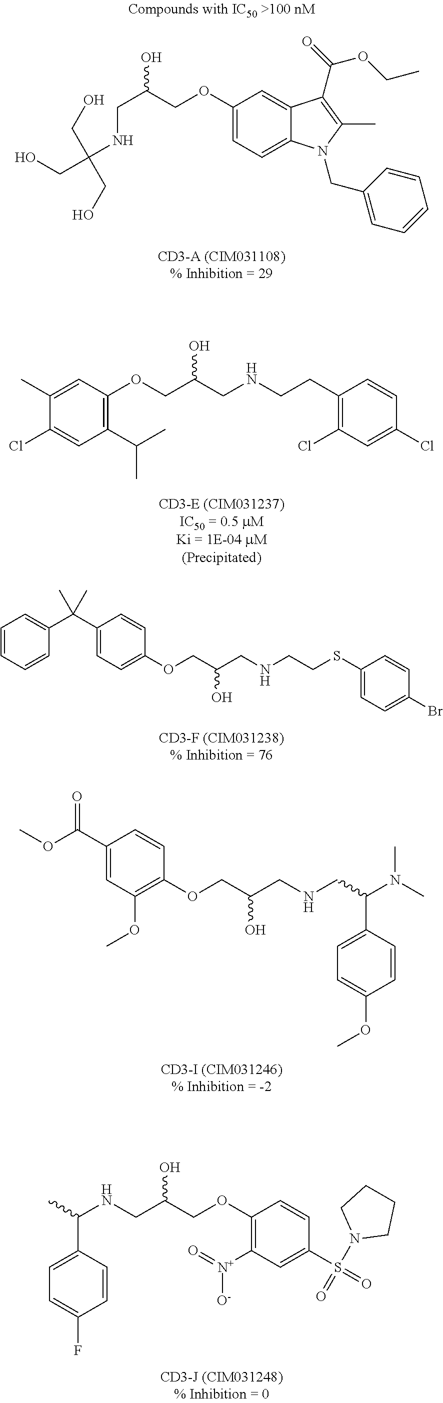

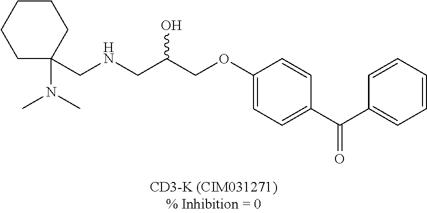 [SMILES: CN(C)C1(CNCC(O)COc2ccc(C(=O)c3ccccc3)cc2)CCCCC1]

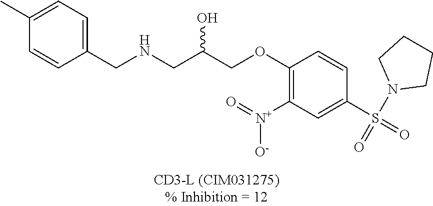 [SMILES: Cc1ccc(CNCC(O)COc2ccc(S(=O)(=O)N3CCCC3)cc2[N+](=O)[O-])cc1]

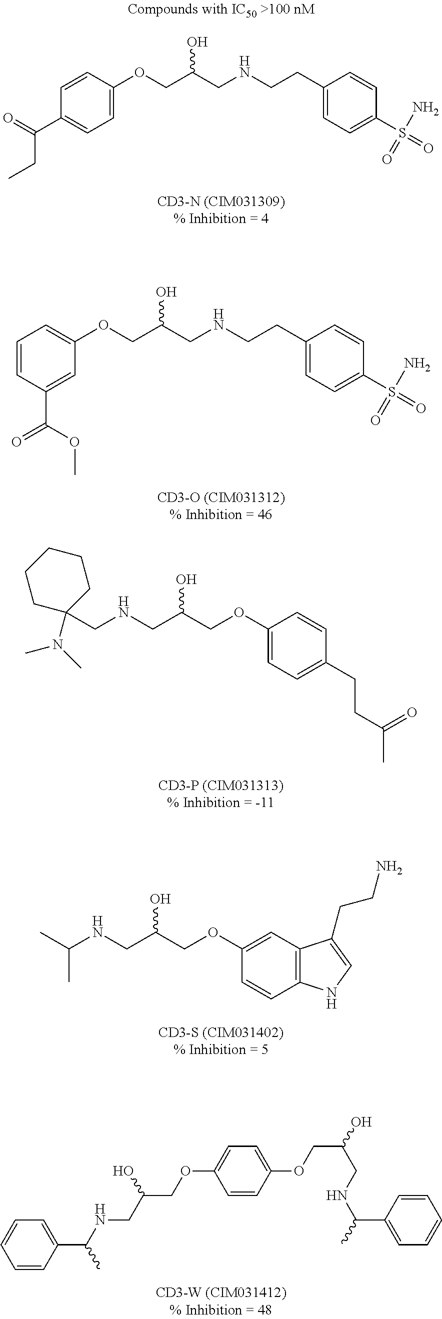

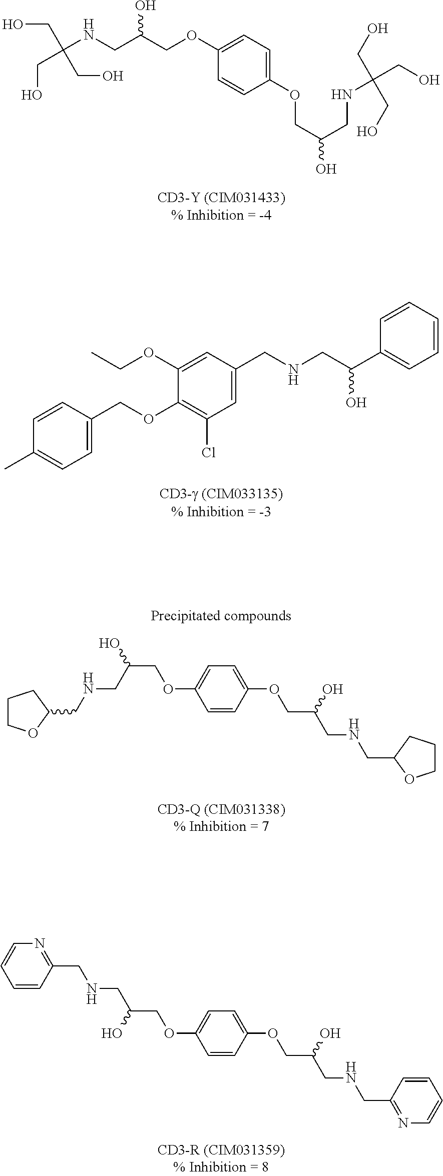 [SMILES: OCC(CO)(CO)NCC(O)COc1ccc(OCC(O)CNC(CO)(CO)CO)cc1 ; CCOc1cc(CNCC(O)c2ccccc2)cc(Cl)c1OCc1ccc(C)cc1 ; OC(CNCC1CCCO1)COc1ccc(OCC(O)CNCC2CCCO2)cc1 ; OC(CNCc1ccccn1)COc1ccc(OCC(O)CNCc2ccccn2)cc1]

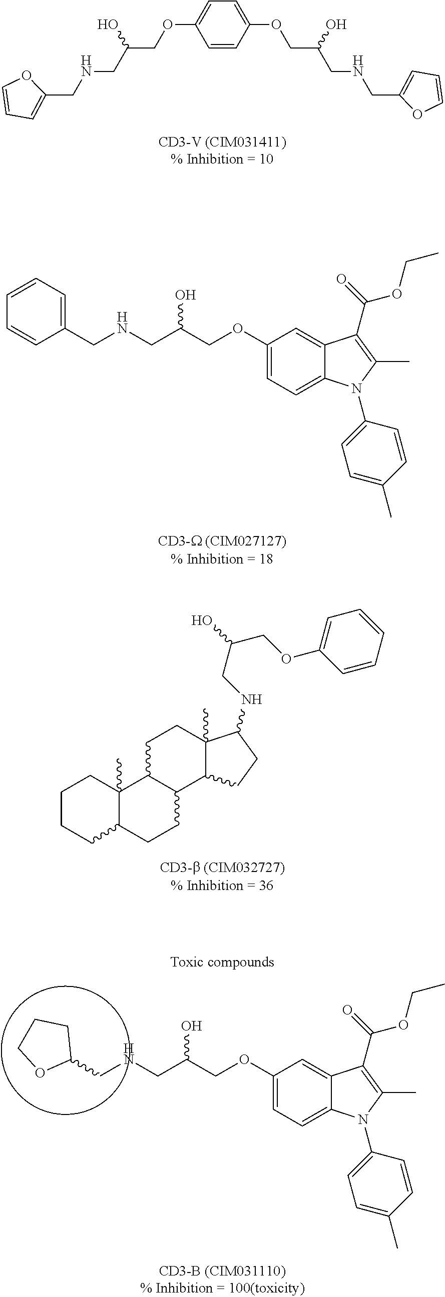

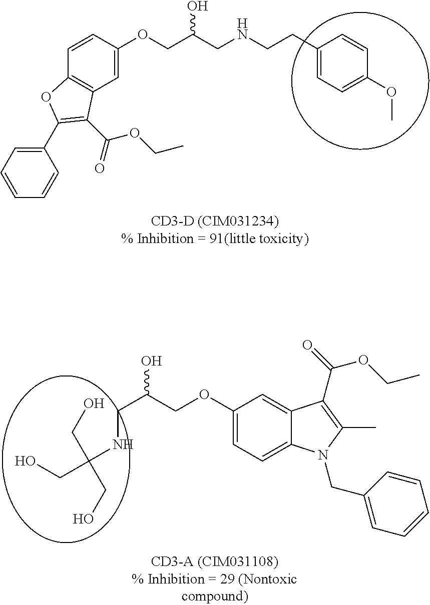 [SMILES: CCOC(=O)c1c(-c2ccccc2)oc2ccc(OCC(O)CNCCc3ccc(OC)cc3)cc12 ; CCOC(=O)c1c(C)n(Cc2ccccc2)c2ccc(OCC(O)CNC(CO)(CO)CO)cc12]

### Cd3 Compounds (Interested)

Concentration response curve CD3-T new and old compound CIM-031403

| Parameter | Value | Std. error |
|---|---|---|
| IC50 (nM)1^{st}, experiment | 221 | 199-244 |
| IC50 (nM)2^{nd}, experiment | 46 | 41-50 |
| IC50 (nM) old stock | 40 | 37-51 |

| | | | A-B permeability | | B-A permeability | | |
|---|---|---|---|---|---|---|---|
| | Client | Test Concen- | (Caco-2, | | (Caco-2, | | |
| STRUCTURE | Compound I.D. | tration (M) | pH 7.4/7.4) | MeanRecovery | pH 7.4/7.4) | MeanRecovery | Efflux |
| 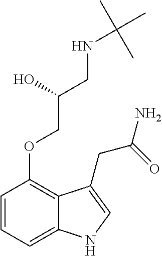 [SMILES: CC(C)(C)NC[C@@H](O)COc1cccc2[nH]cc(CC(N)=O)c12] | CM031408_02_01 | 1.0E−06 | 0.4 | 83 | 1.3 | 94 | 3.25 |

Concentration response curve CD3-T1 (CIM-012783_03_01)

| Parameter | Value | Std. error |
|---|---|---|
| IC50 (nM) 1^{st} expt | 6 | 5-8 |
| IC50 (nM) 2^{nd} expt | 0.6 | 0.56-0.64 |

| | | | A-B permeability | | B-A permeability | | |
|---|---|---|---|---|---|---|---|
| | Client | Test Concen- | (Caco-2, | Mean- | (Caco-2, | Mean- | |
| STRUCTURE | Compound I.D. | tration (M) | pH 7.4/7.4) | Recovery | pH 7.4/7.4) | Recovery | Efflux |
| 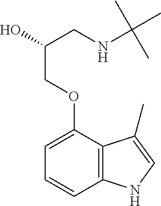 [SMILES: Cc1c[nH]c2cccc(OC[C@H](O)CNC(C)(C)C)c12] | CIM012783_03_01 | 1.0E−06 | 54.7 | 71 | 17.8 | 87 | 0.325411335 |

Concentration response curve CD3-T2 (CIM-121255_01_02)

| Parameter | Value | Std. error |
|---|---|---|
| IC50 (nM) | 3801 | 2733-5248 |

Concentration response curve CD3-T3 (CIM-121256_01_01)

| Parameter | Value | Std. error |
|---|---|---|
| IC50 (nM) 1^{st} expt | 66 | 60-74 |
| IC50 (nM) 2^{nd} expt. | 78 | 70-86 |

| | | Test | A-B permeability | | B-A permeability | | |
|---|---|---|---|---|---|---|---|
| | Client | Concen- | (Caco-2, | Mean- | (Caco-2, | Mean- | |
| STRUCTURE | Compound I.D. | tration (M) | pH 7.4/7.4) | Recovery | pH 7.4/7.4) | Recovery | Efflux |
| 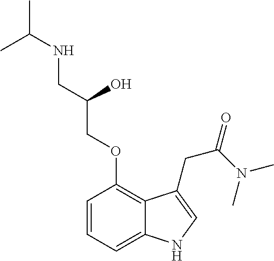 [SMILES: CC(C)NC[C@@H](O)COc1cccc2[nH]cc(CC(=O)N(C)C)c12] | CIM121256_ 01_01 | 1.0E−06 | 0.1 | 76 | 3.2 | 90 | 32 |

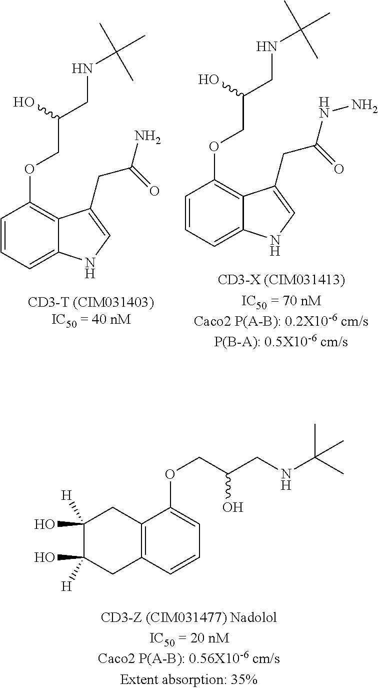

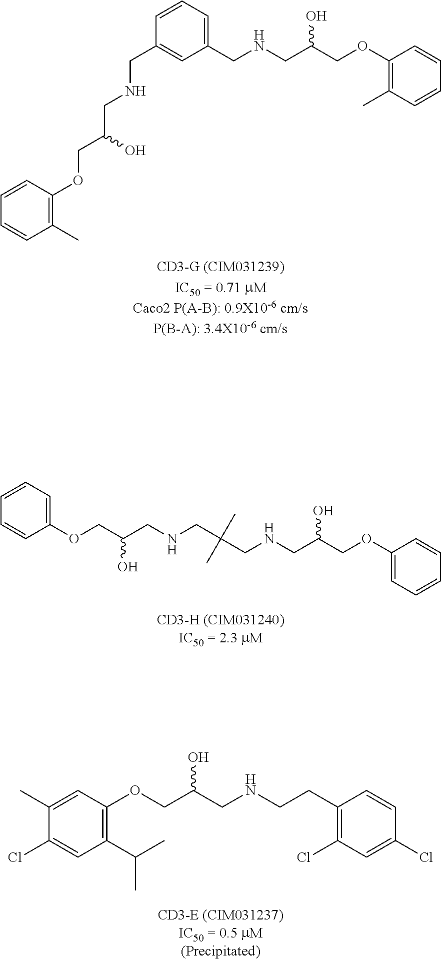 [SMILES: Cc1ccccc1OCC(O)CNCc1cccc(CNCC(O)COc2ccccc2C)c1 ; CC(C)(CNCC(O)COc1ccccc1)CNCC(O)COc1ccccc1 ; Cc1cc(OCC(O)CNCCc2ccc(Cl)cc2Cl)c(C(C)C)cc1Cl]

Initially 30 compounds screened
CD3 X selected (IC50: 70 nM & low permeability in
Caco2 assay) but found to be racemic mixture
Another 3 analogues of CD3T (T1, T2 & T3) tested
CD3T: 46 nM, T1: 6 nM, T2: 3801 nM & T3: 78 nM
CD3T: 0.4 × 10−6 cm/s & CD3T3: 0.1 × 10−6 cm/s have low
permeability due to efflux transporter whereas CD3T1:
54.7 × 10−6 cm/s has good permeability in Caco2 assay

1.-15. (canceled)

16. A method for the treatment of at least one of diabetes and obesity, the method comprising administering an β2-adrenergic receptor (β2-AR) antagonist.

17. The method according to claim 16, wherein the β2-AR antagonist is a non-bioavailable β2-AR antagonist.

18. The method according to claim 16, wherein the β2-AR antagonist is an orally non-bioavailable β2-AR antagonist.

19. The method according to claim 16, wherein the non-bioavailable β2-AR antagonist is a β2-AR antagonist having minimal permeability through biological membranes.

20. The method according to claim 16, wherein the non-bioavailable β2-AR antagonist is a β2-AR antagonist has a permeability through biological membranes that is no more than 2, 1.5, 1.2, 1.0, 0.5, 0.4, 0.2 or 0.1 cm/s, as determined in a Caco-2 cell or other intestinal permeability assay.

21. The method according to claim 16, wherein the non-bioavailable β2-AR antagonist is a non-bioavailable derivative of ICI 118,551, that is modified as compared to ICI 118,551 by at least one of the addition of hydrophilic group, the addition of a bulky group and the elimination of a hydrophobic group and/or by or any other modification that is known by a person skilled in the art to decease oral bioavailability.

22. The method according to claim 16, wherein β2-AR antagonist is a CD3 compound.

23. The method according to claim 16, wherein the CD3 compound is in that it has characterised in at least one of i) having an IC50<100, 50, 20 or 10 nM; ii) lacking toxicity; and iii) being soluble.

24. The method according to claim 22, wherein the CD3 compound is CD3-403 (CIM-031403), CD3-T1 (CIM-012783_03_01), CD3-T3 (CIM-121256_01_01) or a derivative of CD3-403 (CIM-031403), CD3-T1 (CIM-012783_03_01), CD3-T3 (CIM-121256_01_01).

25. The method according to claim 16, wherein the diabetes is Type 1 diabetes or Type 2 diabetes.

26. A non-bioavailable β2-AR antagonist having a permeability through biological membranes that is no more than 2, 1.5, 1.2, 1.0, 0.5, 0.4, 0.2 or 0.1 cm/s, as determined in a Caco-2 cell or other intestinal permeability assay.

27. The non-bioavailable β2-AR antagonist according to claim 26, wherein the non-bioavailable β2-AR antagonist has oral non-bioavailability.

28. The non-bioavailable β2-AR antagonist according to claim 26, wherein the non-bioavailable β2-AR antagonist is a non-bioavailable derivative of ICI 118,551, that is modified as compared to ICI 118,551 by at least one of the addition of hydrophilic group, the addition of a bulky group and the elimination of a hydrophobic group (and/or by or any other modification that is known by a person skilled in the art to decease oral bioavailability).

29. The non-bioavailable β2-AR antagonist according to claim 26, wherein the β2-AR antagonist is a CD3 compound.

30. The non-bioavailable β2-AR antagonist according to claim 29, wherein the CD3 compound is characterised in at least one of i) having an IC50<100, 50, 20 or 10 nM; ii) lacking toxicity; and iii) being soluble.

31. The non-bioavailable β2-AR antagonist according to claim 29, wherein the CD3 compound is CD3-403 (CIM-031403), CD3-T1 (CIM-012783_03_01), CD3-T3 (CIM-121256_01_01) or a derivative of CD3-403 (CIM-031403), CD3-T1 (CIM-012783_03_01), CD3-T3 (CIM-121256_01_01).

32. An assay for identifying a compound that inhibits the sugar induced activation of the β2-AR, the assay comprising the step of contacting a cell expressing β2-AR with a sugar and the compound and determining the activation of the β2-AR, wherein a compound is identified as a compound that inhibits the sugar induced activation of the β2-Arif there is less activation of the β2-AR in the presence of the compound than under corresponding conditions in the absence of the compound.


SEQUENCE LISTING (36 sequences)

SEQ ID NO: 1 - DNA, 23 bp, synthetic construct
  source 1..23: mol_type = other DNA; note = GPR1 fw
tggaccccttatcacctgtttag

SEQ ID NO: 2 - DNA, 20 bp, synthetic construct
  source 1..20: mol_type = other DNA; note = GPR1 re
cctgcagcacattctggaaa

SEQ ID NO: 3 - DNA, 22 bp, synthetic construct
  source 1..22: mol_type = other DNA; note = GPR120 fw
tccgagtgtcccaacaagacta

SEQ ID NO: 4 - DNA, 21 bp, synthetic construct
  source 1..21: mol_type = other DNA; note = GPR120 re
atgatgggactccacatgatg

SEQ ID NO: 5 - DNA, 20 bp, synthetic construct
  source 1..20: mol_type = other DNA; note = GPR125 fw
cgtgcagtttcgaacaaacg

SEQ ID NO: 6 - DNA, 17 bp, synthetic construct
  source 1..17: mol_type = other DNA; note = GPR125 re
ctggcccggtgtccttt

SEQ ID NO: 7 - DNA, 23 bp, synthetic construct
  source 1..23: mol_type = other DNA; note = GPR18 fw
ggagaagtccatacggatcatca

SEQ ID NO: 8 - DNA, 18 bp, synthetic construct
  source 1..18: mol_type = other DNA; note = GPR18 re
gtggaagggcacgaagca

SEQ ID NO: 9 - DNA, 20 bp, synthetic construct
  source 1..20: mol_type = other DNA; note = GPR19 fw
ctaccgcagcaatgcctaca

SEQ ID NO: 10 - DNA, 20 bp, synthetic construct
  source 1..20: mol_type = other DNA; note = GPR19 re
gatttccgaaatgcccacat

SEQ ID NO: 11 - DNA, 23 bp, synthetic construct
  source 1..23: mol_type = other DNA; note = GPR22 fw
tcgtgtttggtgtgagaacttca

SEQ ID NO: 12 - DNA, 16 bp, synthetic construct
  source 1..16: mol_type = other DNA; note = GPR22 re
tcccggtggcgtttca

SEQ ID NO: 13 - DNA, 23 bp, synthetic construct
  source 1..23: mol_type = other DNA; note = GPR39 fw
cccatggagttctacagcatcat

SEQ ID NO: 14 - DNA, 22 bp, synthetic construct
  source 1..22: mol_type = other DNA; note = GPR39 re
cgtgtggagcttacaggacaga

SEQ ID NO: 15 - DNA, 21 bp, synthetic construct
  source 1..21: mol_type = other DNA; note = GPR40 fw
tgcctccaatgtggctagttt

SEQ ID NO: 16 - DNA, 22 bp, synthetic construct
  source 1..22: mol_type = other DNA; note = GPR40 re
ccctgtgatgagtcccaacttc

SEQ ID NO: 17 - DNA, 22 bp, synthetic construct
  source 1..22: mol_type = other DNA; note = GPR48 fw
aagacgactggaagctcctgaa

SEQ ID NO: 18 - DNA, 17 bp, synthetic construct
  source 1..17: mol_type = other DNA; note = GPR48 re
ccgccttggctgctgat

SEQ ID NO: 19 - DNA, 22 bp, synthetic construct
  source 1..22: mol_type = other DNA; note = GPR49 fw
tgagatcaaaacacgcgagtct

SEQ ID NO: 20 - DNA, 18 bp, synthetic construct
  source 1..18: mol_type = other DNA; note = GPR49 re
tgctggcgtgggtaaagg

SEQ ID NO: 21 - DNA, 21 bp, synthetic construct
  source 1..21: mol_type = other DNA; note = GPR56 fw
ccaaggcttcctcatcttcct

SEQ ID NO: 22 - DNA, 22 bp, synthetic construct
  source 1..22: mol_type = other DNA; note = GPR56 re
gcgctgtctgagttgttcttca

SEQ ID NO: 23 - DNA, 22 bp, synthetic construct
  source 1..22: mol_type = other DNA; note = GPR61 fw
aacctggattggctacttttgc

SEQ ID NO: 24 - DNA, 18 bp, synthetic construct
  source 1..18: mol_type = other DNA; note = GPR61 re
taagctcgccccggatct

SEQ ID NO: 25 - DNA, 23 bp, synthetic construct
  source 1..23: mol_type = other DNA; note = GPR80 fw
cctttggcaacctgctgttatat

SEQ ID NO: 26 - DNA, 18 bp, synthetic construct
  source 1..18: mol_type = other DNA; note = GPR80 re
ccgctggctttgcatctc

SEQ ID NO: 27 - DNA, 21 bp, synthetic construct
  source 1..21: mol_type = other DNA; note = GPR85 fw
ttgcacgagggcctgtagtac

SEQ ID NO: 28 - DNA, 20 bp, synthetic construct
  source 1..20: mol_type = other DNA; note = GPR85 re
attcctgcttgggcgaaact

SEQ ID NO: 29 - DNA, 21 bp, synthetic construct
  source 1..21: mol_type = other DNA; note = GPR88 fw
gctctacacgtggaggaacga

SEQ ID NO: 30 - DNA, 17 bp, synthetic construct
  source 1..17: mol_type = other DNA; note = GPR88 re
gttgcgcctgggacatg

SEQ ID NO: 31 - DNA, 22 bp, synthetic construct
  source 1..22: mol_type = other DNA; note = TM7SF1 fw
cccccgaagatatgacagtgat

SEQ ID NO: 32 - DNA, 22 bp, synthetic construct
  source 1..22: mol_type = other DNA; note = TM7SF1 re
ggagcaaaacttccctgaagtc

SEQ ID NO: 33 - DNA, 20 bp, synthetic construct
  source 1..20: mol_type = other DNA; note = TM7SF2 fw
gcagcagtgcctccaaaagt

SEQ ID NO: 34 - DNA, 19 bp, synthetic construct
  source 1..19: mol_type = other DNA; note = TM7SF2 re
ggtaaggcacacgcttgca

SEQ ID NO: 35 - DNA, 20 bp, synthetic construct
  source 1..20: mol_type = other DNA; note = TM7SF3 fw
gaaagaggacagccgccttt

SEQ ID NO: 36 - DNA, 23 bp, synthetic construct
  source 1..23: mol_type = other DNA; note = TM7SF3 re
gatgttggtcactctgcgttctc